SEQUENCE LISTING (17 sequences)

SEQ ID NO: 1 - protein, 353 aa, Homo sapiens
ASPTSPKVFPLSLCSTQPDGNVVIACLVQGFFPQEPLSVTWSESGQGVTARNFPPSQDASGDLYTTSSQLTLPATQCLAGKSVTCHVKHYTNPSQDVTVPCPVPSTPPTPSPSTPPTPSPSCCHPRLSLHRPALEDLLLGSEANLTCTLTGLRDASGVTFTWTPSSGKSAVQGPPERDLCGCYSVSSVLPGCAEPWNHGKTFTCTAAYPESKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 2 - protein, 192 aa, Homo sapiens
  misc_feature (140)..(140): Xaa can be any naturally occurring amino acid
DFPMPFISAKSSPVIPLDGSVKIQCQAIREAYLTQLMIIKNSTYREIGRRLKFWNETDPEFVIDHMDANKAGRYQCQYRIGHYRFRYSDTLELVVTGLYGKPFLSADRGLVLMPGENISLTCSSAHIPFDRFSLAKEGEXSLPQHQSGEHPANFSLGPVDLNVSGIYRCYGWYNRSPYLWSFPSNALELVVT

SEQ ID NO: 3 - protein, 365 aa, Streptococcus pyogenes
MARKDTNKQYSLRKLKTGTASVAVAVAVLGAGFANQTTVKAESSNNAESSNISQESKLINTLTDENEKLREELQQYYALSDAKEEEPRYKALRGENQDLREKERKYQDKIKKLEEKEKNLEKKSEDVERHYLKKLDQEHKEQQERQKNLEELERQSQREIDKRYQEQLQKQQQLETEKQISEASRKSLSRDLEASRAAKKKVEADLAALNAEHQKLKEEKQISDASRQGLSRDLEASREAKKKVEADLAEANSKLQALEKLNKELEEGKKLSEKEKAELQARLEAEAKALKEQLAKQAEELAKLKGNQTPNAKVAPQANRSRSAMTQQKRTLPSTGEAANPFFTAAAATVMVSAGMLALKRKEEN

SEQ ID NO: 4 - protein, 50 aa, Artificial sequence
  Sap
YYALSDAKEEEPRYKALRGENQDLREKERKYQDKIKKLEEKEKNLEKKSC

SEQ ID NO: 5 - protein, 287 aa, Homo sapiens
MDPKQTTLLCLVLCLGQRIQAQEGDFPMPFISAKSSPVIPLDGSVKIQCQAIREAYLTQLMIIKNSTYREIGRRLKFWNETDPEFVIDHMDANKAGRYQCQYRIGHYRFRYSDTLELVVTGLYGKPFLSADRGLVLMPGENISLTCSSAHIPFDRFSLAKEGELSLPQHQSGEHPANFSLGPVDLNVSGIYRCYGWYNRSPYLWSFPSNALELVVTDSIHQDYTTQNLIRMAVAGLVLVALLAILVENWHSHTALNKEASADVAEPSWSQQMCQPGLTFARTPSVCK

SEQ ID NO: 6 - protein, 534 aa, Homo sapiens
MDGEATVKPGEQVPLWTHGWPPDDPSPSFAAGSSFALPQKRPHPRWLWEGSLPSRTHLRAMGTLRPSSPLCWREESSFAAPNSLKGSRLVSGEPGGAVTIQCHYAPSSVNRHQRKYWCRLGPPRWICQTIVSTNQYTHHRYRDRVALTDFPQRGLFVVRLSQLSPDDIGCYLCGIGSENNMLFLSMNLTISAGPASTLPTATPAAGELTMRSYGTASPVANRWTPGTTQTLGQGTAWDTVASTPGTSKTTASAEGRRTPGATRPAAPGTGSWAEGSVKAPAPIPESPPSKSRSMSNTTEGVWEGTRSSVTNRARASKDRREMTTTKADRPREDIEGVRIALDAAKKVLGTIGPPALVSETLAWEILPQATPVSKQQSQGSIGETTPAAGMWTLGTPAADVWITSMEAASGEGSAAGDLDAATGDRGPQATLSQTPAVGPWGPPGKESSVKRATCPGLSTLKEIKVSAVLTQNPHSLLCVSAAQEAERVTLIQMTHFLEVNPQADQLPHVERKMLQDDSLPAGASLTAPERNPGP

SEQ ID NO: 7 - protein, 764 aa, Homo sapiens
MLLFVLTCLLAVFPAISTKSPIFGPEEVNSVEGNSVSITCYYPPTSVNRHTRKYWCRQGARGGCITLISSEGYVSSKYAGRANLTNFPENGTFVVNIAQLSQDDSGRYKCGLGINSRGLSFDVSLEVSQGPGLLNDTKVYTVDLGRTVTINCPFKTENAQKRKSLYKQIGLYPVLVIDSSGYVNPNYTGRIRLDIQGTGQLLFSVVINQLRLSDAGQYLCQAGDDSNSNKKNADLQVLKPEPELVYEDLRGSVTFHCALGPEVANVAKFLCRQSSGENCDVVVNTLGKRAPAFEGRILLNPQDKDGSFSVVITGLRKEDAGRYLCGAHSDGQLQEGSPIQAWQLFVNEESTIPRSPTVVKGVAGGSVAVLCPYNRKESKSIKYWCLWEGAQNGRCPLLVDSEGWVKAQYEGRLSLLEEPGNGTFTVILNQLTSRDAGFYWCLTNGDTLWRTTVEIKIIEGEPNLKVPGNVTAVLGETLKVPCHFPCKFSSYEKYWCKWNNTGCQALPSQDEGPSKAFVNCDENSRLVSLTLNLVTRADEGWYWCGVKQGHFYGETAAVYVAVEERKAAGSRDVSLAKADAAPDEKVLDSGFREIENKAIQDPRLFAEEKAVADTRDQADGSRASVDSGSSEEQGGSSRALVSTLVPLGLVLAVGAVAVGVARARHRKNVDRVSIRSYRTDISMSDFENSREFGANDNMGASSITQETSLGGKEEFVATTESTTETKEPKKAKRSSKEEAEMAYKDFLLQSSTVAAEAQDGPQEA

SEQ ID NO: 8 - protein, 760 aa, Homo sapiens
MMDQARSAFSNLFGGEPLSYTRFSLARQVDGDNSHVEMKLAVDEEENADNNTKANVTKPKRCSGSICYGTIAVIVFFLIGFMIGYLGYCKGVEPKTECERLAGTESPVREEPGEDFPAARRLYWDDLKRKLSEKLDSTDFTSTIKLLNENSYVPREAGSQKDENLALYVENQFREFKLSKVWRDQHFVKIQVKDSAQNSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGTGDPYTPGFPSFNHTQFPPSRSSGLPNIPVQTISRAAAEKLFGNMEGDCPSDWKTDSTCRMVTSESKNVKLTVSNVLKEIKILNIFGVIKGFVEPDHYVVVGAQRDAWGPGAAKSGVGTALLLKLAQMFSDMVLKDGFQPSRSIIFASWSAGDFGSVGATEWLEGYLSSLHLKAFTYINLDKAVLGTSNFKVSASPLLYTLIEKTMQNVKHPVTGQFLYQDSNWASKVEKLTLDNAAFPFLAYSGIPAVSFCFCEDTDYPYLGTTMDTYKELIERIPELNKVARAAAEVAGQFVIKLTHDVELNLDYERYNSQLLSFVRDLNQYRADIKEMGLSLQWLYSARGDFFRATSRLTTDFGNAEKTDRFVMKKLNDRVMRVEYHFLSPYVSPKESPFRHVFWGSGSHTLPALLENLKLRKQNNGAFNETLFRNQLALATWTIQGAANALSGDVWDIDNEF

SEQ ID NO: 9 - protein, 9 aa, Artificial sequence
  IgM binding peptide
YDWIPSSAW

SEQ ID NO: 10 - protein, 273 aa, Staphylococcus aureus
MEQRITLKEAWDQRNGFIQSLKDDPSQSANVLGEAQKLNDSQAPKADAQQNNFNKDQQSAFYEILNMPNLNEAQRNGFIQSLKDDPSQSTNVLGEAKKLNESQAPKADNNFNKEQQNAFYEILNMPNLNEEQRNGFIQSLKDDPSQSANLLSEAKKLNESQAPKADNKFNKEQQNAFYEILHLPNLNEEQRNGFIQSLKDDPSQSANLLAEAKKLNDAQAPKADNKFNKEQQNAFYEILHLPNLTEEQRNGFIQSLKDDPGNSRGSVDLQITN

SEQ ID NO: 11 - protein, 58 aa, Artificial Sequence [sequence omitted]

SEQ ID NO: 12 - protein, 58 aa, Artificial Sequence
  Modified Z-domain
VDNKFNKETIQASQEIRLLPNLNGRQKLAFIHSLLDDPSQSANLLAEAKKLNDAQAPK

SEQ ID NO: 13 - protein, 58 aa, Staphylococcus aureus [sequence omitted]

SEQ ID NO: 14 - protein, 25 aa, Homo sapiens
TVPCPVPSTPPTPSPSTPPTPSPSC

SEQ ID NO: 15 - protein, 627 aa, Homo sapiens [sequence omitted]

SEQ ID NO: 16 - DNA, 2213 bp, Homo sapiens
gctctagaactagtggatcccccgggctgcaggaattctctaaagaagcccctgggagcacagctcatcaccatggactggacctggaggttcctctttgtggtggcagcagctacaggtgtccagtcccaggtgcagctggtgcagtctggggctgaggtgaagaagcctgggtcctcggtgaaggtctcctgcaaggcttctggaggcaccttcagcagctatgctatcagctgggtgcgacaggcccctggacaagggcttgagtggatgggagggatcatccctatctttggtacagcaaactacgcacagaagttccagggcagagtcacgattaccgcggacgaatccacgagcacagcctacatggagctgagcagcctgagatctgaggacacggccgtgtattactgtgcgaaaaccgggatcctggggccgtatagcagtggctggtacccgaactcggactactactactacggtatggacgtctggggccaagggaccacggtcaccgtctcctcagggagtgcatccgccccaacccttttccccctcgtctcctgtgagaattccccgtcggatacgagcagcgtggccgttggctgcctcgcacaggacttccttcccgactccatcactttctcctggaaatacaagaacaactctgacatcagcagcacccggggcttcccatcagtcctgagagggggcaagtacgcagccacctcacaggtgctgctgccttccaaggacgtcatgcagggcacagacgaacacgtggtgtgcaaagtccagcaccccaacggcaacaaagaaaagaacgtgcctcttccagtgattgctgagctgcctcccaaagtgagcgtcttcgtcccaccccgcgacggcttcttcggcaacccccgcagcaagtccaagctcatctgccaggccacgggtttcagtccccggcagattcaggtgtcctggctgcgcgaggggaagcaggtggggtctggcgtcaccacggaccaggtgcaggctgaggccaaagagtctgggcccacgacctacaaggtgaccagcacactgaccatcaaagagagcgactggctcagccagagcatgttcacctgccgcgtggatcacaggggcctgaccttccagcagaatgcgtcctccatgtgtgtccccgatcaagacacagccatccgggtcttcgccatccccccatcctttgccagcatcttcctcaccaagtccaccaagttgacctgcctggtcacagacctgaccacctatgacagcgtgaccatctcctggacccgccagaatggcgaagctgtgaaaacccacaccaacatctccgagagccaccccaatgccactttcagcgccgtgggtgaggccagcatctgcgaggatgactggaattccggggagaggttcacgtgcaccgtgacccacacagacctgccctcgccactgaagcagaccatctcccggcccaagggggtggccctgcacaggcccgatgtctacttgctgccaccagcccgggagcagctgaacctgcgggagtcggccaccatcacgtgcctggtgacgggcttctctcccgcggacgtcttcgtgcagtggatgcagagggggcagcccttgtccccggagaagtatgtgaccagcgccccaatgcctgagccccaggccccaggccggtacttcgcccacagcatcctgaccgtgtccgaagaggaatggaacacgggggagacctacacctgcgtggtggcccatgaggccctgcccaacagggtcaccgagaggaccgtggacaagtccaccgagggggaggtgagcgccgacgaggagggctttgagaacctgtgggccaccgcctccaccttcatcgtcctcttcctcctgagcctcttctacagtaccaccgtcaccttgttcaaggtgaaatgatcccaacagaagaacatcggagaccagagagaggaactcaaaggggcgctgcctccgggtctggggtcctggcctgcgtggcctgttggcacgtgtttctcttcccgcccggcctccagttgtgtgctctcacacaggcttccttctcgaccggcaggggctggctggcttgcaggccacgaggtgggctctaccccacactgctttgctgtgtatacgcttgttgccctgaaataaatatgcacattttatccatg

SEQ ID NO: 17 - protein, 26 aa, Homo sapiens
PLPVIAELPPKVSVFVPPRDGFFGNP

(19) United States
(12) Patent Application Publication
(10) Pub. No.: US 2013/0095036 A1
(43) Pub. Date: Apr. 18, 2013

(54) METHODS AND COMPOSITIONS FOR DIAGNOSIS OF IGA-AND IGM-MEDIATED KIDNEY DISEASES

(71) Applicants: Robert Qiu, Westborough, MA (US); Jiazhou Qiu, Westborough, MA (US); Andrew G. Plaut, Lexington, MA (US)

(72) Inventors: Robert Qiu, Westborough, MA (US); Jiazhou Qiu, Westborough, MA (US); Andrew G. Plaut, Lexington, MA (US)

(73) Assignees: New England Medical Center Hospitals, Inc., Boston, MA (US); RQ Bioscience, Inc., Westborough, MA (US)

(21) Appl. No.: 13/439,473

(22) Filed: Apr. 4, 2012

Related U.S. Application Data
(63) Continuation of application No. 11/989,923, filed on Jun. 15, 2009, now abandoned.
(60) Provisional application No. 60/705,282, filed on Aug. 3, 2005.

(51) Int. Cl. A61K 51/08; A61K 51/10; A61K 49/16; A61K 49/00; A61K 49/14

(52) U.S. Cl. CPC A61K 51/081; A61K 49/00; A61K 49/14; A61K 51/08; A61K 49/16; A61K 51/1078
USPC 424/1.69; 424/9.1; 424/9.34

(57) ABSTRACT

The present invention features noninvasive methods for diagnosing IgA or IgM kidney disorders, such as IgA nephropathy, Henoch-Schönlein purpura, and IgM nephropathy, in a mammal. The invention also features compositions and kits useful in diagnosing these disorders.

### BACKGROUND OF THE INVENTION

The invention relates to the field of diagnostic methods for kidney diseases and compositions and kits useful in the diagnosis of such diseases.

IgA nephropathy (IgAN, Berger's disease) is characterized by the deposition of IgA1 in the mesangium of the renal glomerulus and is the most common glomerulonephritis worldwide. The IgA deposits arise spontaneously, usually in the second or third decade of life and are thought to be immune complexes. The antigen(s) are unknown; IgA itself may be the antigen. The prevalence of the disease is high in the U.S. and Europe, but highest in Asia. The incidence in Japan may be 40-50% of all renal biopsies. Persistent or intermittently detected microscopic hematuria and proteinuria for many years is the clinical feature of this disease, and more than 50% of patients also develop hypertension. It is not a benign illness as once believed, with about 15-40% of patients eventually developing renal failure. Indeed, IgA nephropathy is the main cause of end-stage renal disease in patients with primary glomerular disease that eventually come to renal transplantation.

Henoch-Schönlein purpura (HSP) is another IgA1-mediated post-infectious vasculitis, most often in children ages 2-11 yrs, with kidney damage very similar to IgAN. Prevalance is 22/100,000 under age 14, and 70/100,000 in the group age 4 to 6 years old. HSP is so similar to IgAN that the notion that IgAN is a renal-limited form of HSP has recently gained acceptance (Smith and Feehally, Springer Semin. Immunopathol. 24:477-493, 2003).

IgM nephropathy (IgMN) causes nephrotic syndrome and is characterized by IgM mesangial deposits. It is speculated that these deposits are derived from circulating IgM aggregates or immune complexes, similar to IgAN. IgMN is often seen in children, and the clinical feature of IgMN is very similar to minimal change disease (MCD), in which nephrotic syndrome is the main clinical manifestation, but IgMN results in a significantly higher incidence of hypertension then MCD.

Prior to the present invention, diagnosis of these diseases were by renal biopsy, which removes a sample of the kidney tissue for pathological examination. A renal pathologist does immunofluorescent staining of kidney tissues. This requires sequential application of anti-human IgA antibody, followed by development with a fluorochrome-conjugated second antibody to localize the IgA-based antibody-antigen complexes for the diagnosis of IgAN and HSP. In the diagnosis of IgMN, the same procedures can be applied, but the antibody used specifically binds human IgM. If the test detects high levels of glomerular IgA, more so than co-existing IgG, and with complement components variably present, the diagnosis of IgA nephropathy is made. This is the same for IgMN, in which the deposition of IgM in the glomerulus defines the illness.

Biopsy is usually done with the patient lying in the prone position, the kidney having been localized using ultrasound or CAT scan. Under local anesthesia, a small incision is made in the skin. Using an appropriate breath-holding protocol, a biopsy needle is used to take a sample the size of 1-1.5 cm×2 mm, and the needle is removed. The patient remains in the hospital, lying supine for 12 to 24 hours, with monitoring to detect complications which may include bleeding, pain, arteriovenous fistula, urinary tract infection, and in rare cases, death.

Bleeding is the most common complication of renal biopsy. Although prior to biopsy patients are tested for the ability to coagulate, most patients experience microscopic hematuria for a short time. Rarely, bleeding is severe enough to require a blood transfusion or surgery. It has been estimated that surgery is required to control the bleeding in 0.1 to 0.4 percent of percutaneous renal biopsies, and removal of the kidney to control hemorrhage is required in approximately 0.06 percent (6 per 10,000) of completed needle biopsies.

Pain is a common problem and is transient. Pain lasting more than 12 hours occurs in approximately 4 percent of biopsies. Severe or prolonged pain can occur if a blood clot obstructs one of the ureters or in the event of a large subcapsular hematoma.

Arteriovenous fistula between two blood vessels can result from damage to the walls of an adjacent artery and vein caused by the biopsy needle. Such fistulas are rare and usually close spontaneously in one to two years.

Death occurs in approximately 0.1% of renal biopsy cases.

Renal biopsy is not appropriate for all patients. Contraindications include an uncorrectable bleeding condition, small kidneys, severe hypertension that cannot be medically controlled, multiple bilateral renal cysts or a renal tumor, hydronephrosis (a condition in which the flow of urine is obstructed leading to kidney damage), active infection of the tissues in or surrounding the kidney, inability of the patient to cooperate, and a solitary native kidney. Alternatives to percutaneous biopsy are the open surgery biopsy and transjugular renal biopsy

Thus there is a need for safer, more reliable, and less invasive methods for diagnosing IgA nephropathy, HSP, and IgM nephropathy.

### SUMMARY OF THE INVENTION

The present invention features a method for diagnosing an IgA or IgM kidney disease in a mammal (e.g., a human) which includes administering (e.g., intravenously) to the mammal a compound which specifically binds IgA or IgM and detecting the compound in the kidney of the mammal, where an increase in the amount of the compound in the kidney of the mammal relative to the amount in the kidney of a mammal without the kidney disease is diagnostic of the IgA or IgM kidney disease in the mammal.

The compound may include a peptide (e.g., human FcαR1 (SEQ ID NO:2), human Fcα/μ receptor (SEQ ID NO:6), polymeric Ig receptor (SEQ ID NO:7), Sir22 (SEQ ID NO:3), streptococcal IgA-binding peptide (Sap; SEQ ID NO:4), a modified Z-domain protein (SEQ ID NO:12), and YDWIPSSAW (SEQ ID NO:9), or an IgA- or IgM-binding fragment of these peptides). The peptide may include a modification of an N-terminal cap, a C-terminal cap, a D-amino acid, an amino acid surrogate, a peptidomimetic sequence, or a spacer. The compound may include an antibody or an IgA- or IgM-binding fragment of an antibody (e.g., anti-human IgA or anti-human IgM). The compound may also include a qdot. The compound may be linked to a a radioactive label (e.g., ^{99m}Tc, ¹¹¹In, ⁶⁶Ga, ⁶⁷Ga, ⁶⁸Ga, ⁸⁶Y, ⁹⁰Y, ²⁰¹Tl, ⁵⁵Co, ⁶⁰Cu, ⁶¹Cu, ⁶²Cu, ⁶⁴Cu, ⁶⁷Cu, ⁸²Rb, ^{185/187}Re, and ^{186/188}Re). The linkage may be by a bifunctional chelating agent, which may include N₃S, N₂S₂, PnAO, HYNIC, [M(CO)₃(H₂O)₃]⁺, PADA, DTPA, DOTA, histidine, a tripeptide (e.g., Lys-Gly-Cys, Cys-Gly-Cys, and Gly-Gly-Cys), and a tetrapeptide (e.g., Gly-Ala-Gly-Gly or Cys-Gly-Cys-Gly). The compound may be linked to a paramagnetic substance (e.g., gadolinium). The detection may be carried out by an imaging technique (e.g., SPECT, PET, planar scan and MRI).

In a further embodiment, the compound includes a galactose and a first member of a binding pair (e.g., streptavidin). The administration of the compound may further include administration of galactose-Ficoll. The detection may then be performed by administering to the mammal a radioactively labeled (e.g., ^{99m}Tc, ¹¹¹In, ⁶⁶Ga, ⁶⁷Ga, ⁶⁸Ga, ⁸⁶Y, ⁹⁰Y, ²⁰¹Tl, ⁵⁵Co, ⁶⁰Cu, ⁶¹Cu, ⁶²Cu, ⁶⁴Cu, ⁶⁷Cu, ⁸²Rb, ^{185/187}Re, or ^{186/188}Re labeled) compound (e.g., human serum albumin) conjugated to a second member of a binding pair (e.g., biotin) and a galactose followed by detecting the radioactively labeled compound in the kidney of the mammal.

A second aspect of the invention is a composition including an IgA- or IgM-binding compound, a bifunctional chelating agent, and a detectable label in a pharmaceutically acceptable carrier, where the IgA- or IgM-binding compound is linked to the detectable label through the bifunctional chelating agent. The compound may include a peptide (e.g., human FcαR1 (SEQ ID NO:2), human Fcα/μ receptor (SEQ ID NO:6), polymeric Ig receptor (SEQ ID NO:7), Sir22 (SEQ ID NO:3), streptococcal IgA-binding peptide (Sap; SEQ ID NO:4), a modified Z-domain protein (SEQ ID NO:12), and YDWIPSSAW (SEQ ID NO:9), or an IgA- or IgM-binding fragment of one of these peptides). The peptide may include a modification of an N-terminal cap, a C-terminal cap, a D-amino acid, an amino acid surrogate, a peptidomimetic sequence, or a spacer. The compound may also include an antibody (e.g., anti-human IgA or anti-human IgM), or an IgA- or IgM-binding fragment of the antibody. The bifunctional chelating agent may be N₃S, N₂S₂, PnAO, HYNIC, [M(CO)₃(H₂O)₃]⁺, PADA, DTPA, DOTA, histidine, a tripeptide (e.g., Lys-Gly-Cys, Cys-Gly-Cys, or Gly-Gly-Cys.), or a tetrapeptide (e.g., Gly-Ala-Gly-Gly or Cys-Gly-Cys-Gly).

In a third aspect, the invention provides a kit including an IgA- or IgM-binding compound, a bifunctional chelating agent; a detectable label in a pharmaceutically acceptable carrier; and instructions for use for detecting an IgA or IgM kidney disease in a mammal. The compound may include a galactose and a first member of a binding pair (e.g., streptavidin); and the detectable label may include a radioactively labeled peptide (e.g., human serum albumin), the radioactively labeled (e.g., ^{99m}Tc, ¹¹¹In, ⁶⁶Ga, ⁶⁷Ga, ⁶⁸Ga, ⁸⁶Y, ⁹⁰Y, ²⁰¹Tl, ⁵⁵Co, ⁶⁰Cu, ⁶¹Cu, ⁶²Cu, ⁶⁴Cu, ⁶⁷Cu, ⁸²Rb, ^{185/187}Re, or ^{186/188}Re labeled) peptide including a galactose and a second member of a binding pair (e.g., biotin).

By “IgA or IgM kidney disease” is meant a disorder of the kidney mediated by IgA or IgM deposition in the kidney. These disorders include, as examples, IgA nephropathy, Henoch-Schönlein purpura, and IgM nephropathy.

By “specifically binds” is meant a compound which recognizes and binds, a compound, for example, IgA or IgM, but which does not substantially recognize and bind other molecules in a sample, for example, a biological sample, which naturally includes that compound. In one example, an antibody that specifically binds to IgA1 (SEQ ID NO:1) recognizes a 13 amino acid region present in IgA1 and absent in IgA2. Desirably, the compound binds the compound of interest, for example, IgA, at least 5-fold, 10-fold, 25-fold, 50-fold, 100-fold, or 1000-fold more strongly than it binds other components of the sample.

By “N-terminal cap” is meant any chemical modification to the amino-terminal end of a peptide or protein. In the present invention, the addition of an N-terminal cap to a peptide or protein is intended to decrease the rate of in vivo degradation of the peptide or protein as compared to the uncapped protein. Examples of N-terminal caps include acetylation and peptide cyclization.

By “C-terminal cap” is meant any chemical modification to the carboxy-terminal end of a peptide or protein. In the present invention, the addition of a C-terminal cap to a peptide or protein is intended to decrease the rate of in vivo degradation of the peptide or protein as compared to the uncapped protein. Examples of C-terminal caps include amidating or reducing the C-terminus, and peptide cyclization.

By “peptidomimetic” is meant a molecule that mimics characteristics of peptides, including the ability to recognize biomolecules. In the present invention, peptidomimetics are inserted into peptides or proteins to prevent in vivo degradation by endopeptidases and exopeptidases.

By “spacer” is meant a small molecule that is inserted in place of an amino acid in a peptide sequence either internally or at either the N- or C-termini. An examples of a spacer includes aminohexanoic acid. Like peptidomimetics, spacers are used in the present invention to prevent peptide degradation.

By “amino acid surrogate” is meant any chemical compound that can be placed into a peptide or protein in place of an amino acid. Examples of amino acid surrogates include spacers, peptidomimetics, imino acids, and amino acids with methylated amide and/or methylated side chain nitrogens.

By “fragment” is meant a chain of at least 4, 5, 6, 8, 10, 15, 20, or 25 amino acids or nucleotides which comprises any portion of a larger peptide or polynucleotide.

By “peptide” is meant any chain of amino acids, or analogs thereof, regardless of length or post-translational modification (for example, glycosylation or phosphorylation).

By “qdot” is meant a fluorescent semiconductor nanocrystal. Example of materials from which qdots are made include CdS, CdSe, CdTe, CdHgTe/ZnS, InP, InAs, and PbSe.

Other features and advantages of the invention will be apparent from the following Detailed Description, the drawings, and the claims.

### BRIEF DESCRIPTION OF THE DRAWINGS

FIG. 1 is a diagram of dimeric IgA1 (SEQ ID NO:1), its hinge region (SEQ ID NO:14), and the O-glycan sites (Thr225, Thr228, Ser230, Ser232, Thr236).

FIG. 2 is a diagram of the structure and characteristics of the Fc-receptor for IgA (FcαR1/CD89; SEQ ID NO:2). The extracellular domains 1 and 3 (EC-1 and EC-2) as well as the associated pair of γ-chains with their signaling (ITAM) motifs are depicted. Furthermore the characteristics of CD89, its cellular distribution, and its known functions are summarized. (From: Westerhuis, Pathogenetic Aspects of IgA—Nephropathy 2001 PrintPartners Ipskamp, Enschede)

FIG. 3 is a schematic representation of the streptococcal Sir22 (M22; SEQ ID NO:3) protein and sequence of the Sap peptide (SEQ ID NO:4) derived from this protein. The Sap peptide includes the 29-residue IgA-binding region (horizontal arrows labeled “IgA”) and the 10 residues on either side of this region. In addition, Sap includes a C-terminal cysteine residue not present in Sir22 that promotes its dimerization. The regions in Sir22 known to include binding sites for C4BP (the human complement regulator), IgA, and IgG are indicated. The C4BP binding region is a hypervariable domain. (The horizontal arrows under designated “C4BP” and “IgG” indicate corresponding regions for binding C4BP and IgG. There are some sequence overlaps for each of these three binding regions). The position of the conserved C repeat region is also indicated. The C-terminal end of Sir22 is covalently bound to peptidoglycan in the bacterial cell wall.

FIG. 4 is a schematic representation of an intact Ig together with Fab and Fv fragments and single V (colored ovals; dots represent antigen-binding sites) and C domains (uncolored). Engineered recombinant antibodies are shown as scFv monomers, dimers (diabodies), trimers (triabodies), and tetramers (tetrabodies), with linkers represented by a black line. Minibodies are shown as two scFv modules joined by two C domains. Also shown are Fab dimers (conjugates by adhesive polypeptide or protein domains) and Fab trimers (chemically conjugated). Colors denote different specificities for the bispecific scFv dimers (diabodies) and Fab dimers and trimers.

FIG. 5 is a diagram of structures of bifunctional chelating agents (BFCAs) for ^{99m}Tc. Triamidethiols (N₃S), diamidedithiols (N₂S₂), propyleneamineoxime (PnAO), and hydrazinonicotinic acid (HYNIC) are shown.

FIG. 6 is a diagram of structures of picolylamine-N,N-diacetic acid (PADA) and its complex with Tc. PADA and its complex after reaction with the aquaion [Tc(CO)₃(H₂O)₃]⁺ are shown.

FIG. 7 is a diagram of structures of BFCAs for ¹¹¹In. Diethyl-enetriaminepentaacidic acid (DTPA) and tetraazacyclo-dodecanetetraacidic acid (DOTA) are shown.

FIG. 8 is a list of amino acid and nucleic acid sequences including IgA1 C-region (SEQ ID NO:1), CD89 (SEQ ID NO:2), Sir22 (SEQ ID NO:3), Sap (SEQ ID NO:4), soluble CD89 (SEQ ID NO:5), Human Fcα/μR (SEQ ID NO:6), pIgR (SEQ ID NO:7), CD71 (SEQ ID NO:8), IgM binding peptide (SEQ ID NO:9), Staph Protein A (SEQ ID NO:10), Z-domain (SEQ ID NO:11), modified Z-domain (SEQ ID NO:12), B-domain (SEQ ID NO:13), the IgA1 hinge region (SEQ ID NO:14), IgM mu chain amino acid sequence (SEQ ID NO:15), IgM mu chain nucleic acid sequence (SEQ ID NO:16), and the IgM hinge sequence (SEQ ID NO:17).

### DETAILED DESCRIPTION

The present invention uses radiologic scans to identify patients who have glomerular-based renal diseases, such as IgA nephropathy (IgAN), Henoch-Schönlein purpura (HSP), and IgM nephropathy (IgMN). IgAN is characterized by a time-dependent (years) deposition of IgA1 immunoglobulins (SEQ ID NO:1) into the glomeruli of the kidney cortex. HSP is closely related to IgAN, with the same pattern of IgA1 deposition and kidney injury. IgMN is characterized by IgM deposition in the mesangium of glomeruli. To identify those patients with renal disease who have IgA1 or IgM deposition, it has been necessary to carry out a closed needle biopsy of the kidney, a somewhat painful procedure of moderate risk that yields a tissue core for pathological examination. With the present invention IgA1 or IgM deposition can be diagnosed using radiologic imaging methods in which the IgA or IgM deposits in the kidney cortex are detected using an injected, labeled IgA-binding or IgM-binding molecule.

When one biopsies the kidney in order to make the diagnosis of kidney disease, the key criteria for diagnosis of IgAN, HSP, and IgMN is the presence of dominant IgA1 or IgM deposits in the kidney glomeruli, the only site of IgA or IgM deposition. As normal persons and those with non-IgAN, non-IgM kidney diseases do not have significant amounts of IgA1 or IgM in their renal glomeruli, the present invention represents a macro-scale, non-invasive detection method for IgA or IgM in the kidney cortex. Another advantage of the present invention is that scanning provides information about the entire kidney cortices in both kidneys, thus reducing sampling error inherent in needle biopsy methods (Sund et al., Nephrol. Dial. Transplant. 14:2445-2454, 1999).

The invention features several IgA-specific peptides that target IgA1, available radioisotopes by which they may be labeled, and linkers used for this labeling. In one embodiment, radionuclide-labeled IgA-binding peptides or proteins are used as diagnostic radiopharmaceuticals to detect IgA deposits in the kidney. The injected radiolabeled IgA-binding peptides bind to IgA throughout the body. Because there is little IgA deposited in the glomeruli of a healthy kidney, IgA deposits in the glomeruli of patients with IgAN result in a high concentration of the emission of radiation from the cortex of the kidney, where the glomeruli are located. This radiation can be detected by various nuclear imaging techniques and therefore may be used to diagnose IgAN. For diagnosis of the kidney disease, antibodies of human origin, or antibodies that are humanized, or animal monoclonal antibodies with specificity for human IgA1 are well-suited as IgA binding compounds, and can also be used to detect IgA1 deposits in the glomeruli.

Additional types of nephropathies including IgM nephropathy can be diagnosed similarly. Replacing an IgA-binding protein or antibody with an anti-human IgM antibody or its fragment, for example, IgM can be detected in IgM nephropathy.

### IgA Structure

Human immunoglobulin A (IgA) synthesis exceeds the combined total of all the other immunoglobulin classes (Rifai et al., J. Exp. Med. 191:2171-2181, 2000). It is estimated that 66 mg of IgA/kg of body weight is produced every day, compared with 34 mg of IgG and 7.9 mg of IgM. There are two isotypes of IgA, IgA1 (SEQ ID NO:1) and IgA2. On mucosal surfaces (e.g., gut, respiratory tract, genital tract) both IgA1 and IgA2 are present, synthesized by local B cells. In the blood, however, IgA1 predominates, produced by B cells in the bone marrow, lymph nodes, and spleen (Donadio and Grande, N. Engl. J. Med. 347:738-748, 2002).

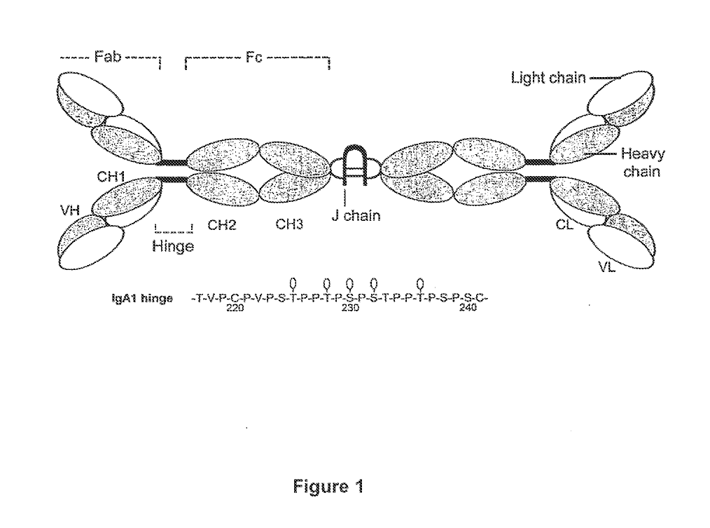

The main difference between the IgA1 (SEQ ID NO:1) and IgA2 subclasses is a 13 amino acid deletion in the IgA2 hinge region. This segment in IgA1 contains several serine and threonine amino acid residues that are O-glycosylated. The absence of this region in IgA2 explains the lack of O-linked oligosaccharides on IgA2 (FIG. 1).

### IgM Structure

IgM (SEQ ID NO:15) can be found as a monomer on the surface of the B lymphocyte, but in circulation it exists mainly as a pentamer after being secreted from plasma cells. The IgM pentamer has a molecular mass of ˜850-1,000 kDa while each monomer is ˜180 kDa. IgM represents ˜10% of total serum Ig and is the first isotype of antibody synthesized during a primary humoral response.

The normal plasma IgM level in adults is about 1 mg/ml with a half-life about 5 days. This compares with IgG level of 12 mg/ml with 25 days of half-life and IgA of 1.8 mg/ml with half-life of 6 days.

Carbohydrates constitute about 12% of the IgM protein by weight. The mu heavy chain of IgM consists of 4 CH domains. (Only mu and epsilon (IgE) heavy chains each have four constant heavy region domains—CH1, CH2, CH3 & CH4, while gamma (IgG), alpha (IgA), and delta (IgD) each have 3 constant heavy region domains.)

Due to the presence of 10 identical antigen-binding sites on a pentamer, IgM is an excellent agglutinating antibody. In addition, IgM is also efficient at activating complement. The IgM monomers are joined together as a pentamer through interchain disulfide bonds, and also by J chain, a 15 kDa peptide that attaches to two monomers of IgM and places the pentamer into a closed, apparently circular, conformation. (J chain also serves to link monomers of IgA together, forming dimers.)

### Diagnostic Compositions

The components of the diagnostic radiopharmaceutical for use herein include (1) a compound, peptide, or protein that specifically binds human IgA or IgM and (2) a compound (e.g., a radioisotope) capable of detection by a radiologic imaging technique (e.g., SPECT) chelated to the compound, peptide, or protein in a pharmaceutically acceptable carrier. Preferable agents include a bifunctional chelating agent (BFCA), which is used to chelate (1) and (2).

While any IgA-binding or IgM-binding compound, protein, or peptide may be used as a biomolecule for diagnosis, preferred candidates meet several or all of the following criteria: high specificity and affinity for human IgA or IgM; for IgA-mediated disorders, ability to differentiate IgA1 from IgA2; small molecules, as these are less immunogenic; a protein of human origin; easily expressed or chemically synthesized; easy to produce, and modify; and appropriately glycosylated, containing galactose for asialoglycloprotein receptor (ASGPR) clearance thereby minimizing renal tubule retention.

### IgA- and IgM-binding Compounds/Peptides

IgA- and IgM-binding proteins or peptides can include native human IgA-binding receptors on cells; receptors that recognize both IgA and IgM; bacterial IgA-binding proteins and their derived peptides; anti-human IgA or IgM antibodies and their smaller derivatives (e.g., Fab); and IgA- and IgM-specific peptides discovered using methods such as phage display.

### IgA-Specific Receptors

The natural functions of IgA-specific receptors include recruitment of inflammatory cells and mediators to inflammatory sites (CD89; SEQ ID NO:2), and the distribution of IgA, e.g., entry of IgA into secretions (pIgR; SEQ ID NO:7).

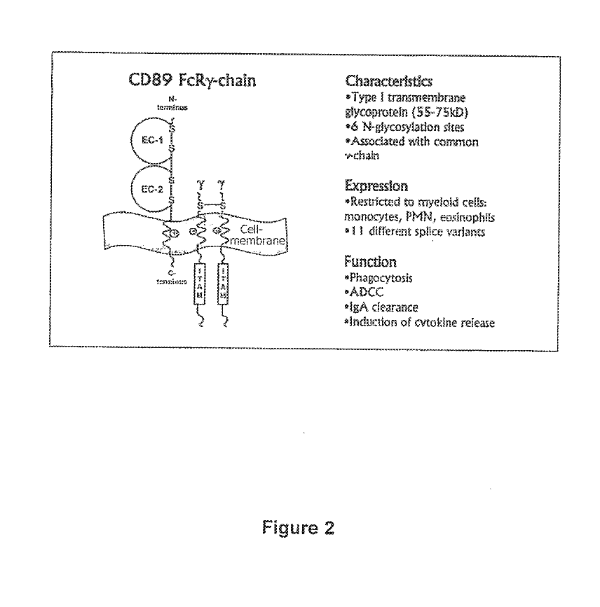

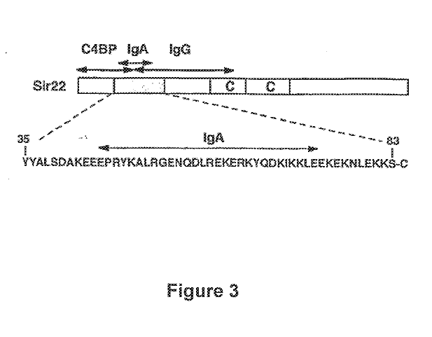

Human FcαR1 (CD89; SEQ ID NO:2; see FIG. 2) is a membrane glycoprotein that contains two extracellular Ig-like domains (206 aa), a membrane-spanning region (19 aa), and a cytoplasmic tail (31 aa). At the transmembrane region, a positively charged arginine residue is necessary for association of CD89 with the FcR γ-chain. Like other Fc receptors lacking an intracellular signaling motif, FcαR's signaling into the cell is initiated via its association with FcR γ-chain. The FcR γ-chain is a homodimer-signaling unit with a size of 10 kDa. Binding to CD89 leads to phosphorylation of the intracellular, immunoreceptor tyrosine-based activation motif (ITAM) on the γ-chain, activating the signaling pathways downstream into the cell. FcαR1 binds both the monomeric and dimeric forms of IgA1 and IgA2. Transfection studies in leukocytes show that the FcαR1 does not bind IgG. FcαR1 is expressed only by myeloid cells, including neutrophils, monocytes, macrophages, and eosinophils. It was proposed that FcαR1 plays a role in the removal of IgA-antigen complexes from the circulation (Mattu et al., J. Bio. Chem. 273:2260-2272, 1998; Leung et al., J. Am. Soc. Nephrol. 11:241-249, 2000; Westerhuis, Pathogenetic Aspects of IgA—Nephropathy 2001, Chapter 1, Section IV: IgA receptors and IgAN, 2001, PrintParters, Ipskamp, Enschede).

A 206 amino acid soluble portion of recombinant CD89 (SEQ ID NO:5) has been successfully expressed in several research labs, and such a soluble receptor has the potential to be used as an IgA-detecting peptide for diagnosis of IgAN. Despite some controversial reports that soluble CD89 might exacerbate IgAN (Pierre Launay et al., J. Exp. Med. 191:1999-2009), this fragment is preferred for binding to IgA due to its high specificity and affinity. This protein is glycosylated, which likely accelerates its clearance via the asialoglycoprotein receptor (ASGPR). This minimizes background noise of unbound ligand in circulation and likely minimizes the clearance of radioactive nuclides through the renal degradation system, which also reduces background noise. More preferably, a smaller fragment of this peptide that retains IgA-binding activity is used.

### Receptors Specifically Binding Both IgA and IgM

The human Fcα/μ receptor (Fcα/μ R; SEQ ID NO:6) binds both IgA and IgM with intermediate to high affinity. Fcα/μ R is constitutively expressed on the majority of B-lymphocytes and macrophages (Sakamoto et al., Eur. J. Immunol. 31:1310-1316, 2001; Shibuya et al., Nat. Immunol. 1:441-446, 2000).

The polymeric Ig receptor (pIgR; SEQ ID NO:7) is an integral membrane component localized on the basolateral surface of secretory epithelial cells. It mediates the trans-epithelial transport of polymeric Ig, mainly dimeric IgA and pentameric IgM. pIgR is on most human secretory epithelia, including intestine, bronchus, salivary glands, renal tubule, and uterus, and it binds to J chains on polymeric immunoglobulins. Binding of IgA results in the protein being transferred from the lamina propria of the mucosa through the epithelial cell to the gut (or other IgA secretory sites) to reach the cell-free fluids bathing the mucus membranes. Secretory IgA (sIgA) is responsible for neutralization of microbes and toxins and prevents unwanted antigens from passing through the mucosal barrier (Mattu et al., J. Bio. Chem. 273:2260-2272, 1998; Leung et al., J. Am. Soc. Nephrol. 11:241-249, 2000).

Recently, other IgA receptors have been proposed, including the transferrin receptor (CD71; SEQ ID NO:8) expressed on mesangial cells (Haddad et al., J. Am. Soc. Nephrol. 14:327-337, 2003). Although the role of this protein in IgA1 deposition diseases is unknown, it may also be used in the present invention.

### Bacterial IgA-Binding Peptides

Bacterial surface proteins that bind human IgA-Fc have been described in both group A streptococci (Streptococcus pyogenes) and group B streptococci (Streptococcus agalactiae; Sandin et al., J. Immunol. 169:1357-1364, 2002; Pleass et al., J. Biol. Chem. 276:8197-8204, 2001; Johnsson et al., J. Biol. Chem. 274:14521-14524, 1999; Stenbere et al., J. Biol. Chem. 269:13458-13464, 1994). The IgA-binding proteins of S. pyogenes are members of the M protein family, a heterogeneous family of dimeric proteins that are important virulence factors. All M proteins bind one or more human plasma proteins, and about 50% of all S. pyogenes strains express an M protein that binds IgA-Fc. M proteins have structural features that foster IgA binding, for example, in several instances a heptad repeat pattern forms a coiled-coil dimer, which binds to specific sites on IgA. Protein Sir22 (also named protein M22, as it is among the M proteins; SEQ ID NO:3), which has been studied in detail by Sandin et al. (J. Immunol. 2002, 169:1357-1364), has such a structure with a stretch of 29 amino acids sufficient for IgA binding. A 50-residue synthetic peptide (SEQ ID NO:4) has been designed that includes the 29-residue IgA-binding region and which specifically binds to human IgA. This 50-mer, designated Streptococcal IgA-binding peptide or Sap (SEQ ID NO:4), has the properties of an isolated IgA-binding domain, and its binding site on IgA is known to be in the Fc region, the same site that binds human CD89. Sap is a homologue of amino acids 35-83 of Sir22 and was designed to include a C-terminal cysteine residue not present in Sir22. The cysteine was introduced to bring about dimerization of the Sap peptide, a process that can be enhanced by incubation with CuCl₂. The IgA-binding tests conducted by Lindahl et al. indicate that Sap dimerization is essential for IgA binding. Sap peptide immobilized on a solid support has been shown to deplete all isotypes, monomers and polymers of IgA from human serum, and eluates from Sap chromatographic columns contain only IgA. Thus, dimerized Sap represents a preferred IgA-binding peptide for IgAN diagnosis, having both IgA-binding specificity and apparent high affinity. Additionally, Sap can be synthesized by solid phase peptide synthesis (SPPS) methods, and a bifunctional chelating agent (BFCA; see below for details) can be added to either the N- or C-terminus while the residue is still in the solid phase on the resin, using methods standard in the art. Sap analogs are small (about 5.5 kDa for a monomer and 11 kDa when dimerized), thus minimizing antigenicity.

In Staphylococcus aureus protein A (SPA; SEQ ID NO:10), one can find five Ig binding domains. SPA binds strongly to the Fc region of immunoglobulins. Z-domain (SEQ ID NO:11) is a 58-aa residue recombinant protein derived from one of these five homologous domains (the B domain; SEQ ID NO:13) in SPA. Z-domain was originally developed by changing a few amino acids of the B domain to enhance the stability of the latter as a gene-fusion partner for affinity purification of recombinant proteins by using IgG-containing resins. Using phage display technology, researchers at Karolinska Institute in Sweden modified the IgG-binding Z-domain into an IgA-binding peptide designated as Affibody_{IgA1} or modified Z-domain (SEQ ID NO:12). (Graille et al., Proc. Natl. Acad. Sci. U.S.A. 97:5399-5404, 2000; Wahlberg et al., Proc. Natl. Acad. Sci. U.S.A. 100:3185-3190, 2003; Rönnmark et al., Eur. J. Biochem. 269: 2647-2655, 2002; Braisted and Wells, Proc. Natl. Acad. Sci. U.S.A. 93:5688-5692, 1996). This peptide binds both human IgA1 and IgA2 with high specificity and affinity. The original IgG binding affinity was completely lost with these modifications. The binding site on IgA is believed to be in the Fe region. Given its binding to human IgA, this peptide is also preferred for IgAN diagnosis.

### Anti-Human IgA or IgM Antibodies or Their Antigen-Binding Fragments

Polyclonal or monoclonal anti-human IgA or IgM antibodies may also be used for the diagnosis of IgA or IgM kidney diseases. These antibodies may be derived from many sources including immunized animals or they may be prepared as animal/human chimeric proteins or humanized chimeric proteins, or human origin protein; all are strategies known in the art for making protein infusion therapies and/or diagnostic reagents. Both whole molecules and Ag-binding fragments of these antibodies can be used in the diagnostic methods and compositions of the invention. However, a preferred reagent is a monoclonal antibody of human origin, and, in the case of IgAN or HSP, preferably one that differentiates IgA1 from IgA2. Also, it is preferred that subunits of the antibody protein, which are preferred over the full-length antibody, maintain IgA- or IgM-recognition specificity (Reff et al., Canc. Control 9:152-166, 2002; Gorman and Clark, Semin. Immunol. 2:457-66, 1990; Antibodies as Medicines 2000 by Biotech Analytics).

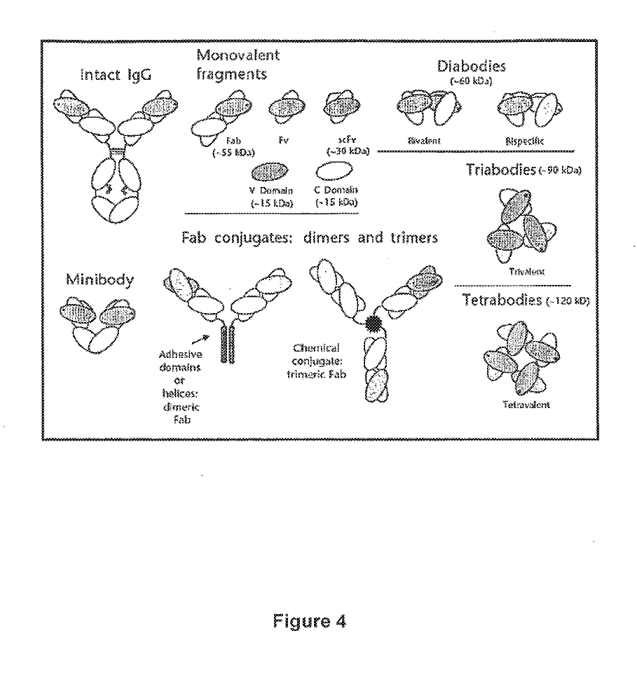

In a preferred embodiment, small antibody fragments of anti-human IgA1 or anti-human IgM (e.g., human-originated fragments, humanized chimeric fragments, chimeric fragments, and animal origin fragments) are used. The fragments of the antibody that retain antigen-binding activity may be monovalent Fab, Fv, or scFv; or bivalent F(ab)′2 or diabodies. (See the schematic diagrams (Hudson and Souriau, Nat. Med. 9:129-134, 2003) in the FIG. 4 below). Preferably, the Fc region is deleted from the molecule.

In one particular example, the anti-human IgA1 antibody is directed at epitopes in the hinge region because, as described above, this region is unique to IgA1. Such human origin anti-human IgA1 hinge region Fab may be readily made by phage display technology through a large phage antibody library developed by commercial providers such as Cambridge Antibody Technology (Cambridge, UK). This technology has advantages over methods for raising conventional monoclonal antibodies in being rapid, and in allowing access to the gene for ease of modification.

In another example, anti-IgM monoclonal antibodies, raised using methods standard in the art (e.g., those described herein), are used to diagnose IgMN. These antibodies can be raised using the unique mu chain hinge sequences that bridge CH1 and CH2 domains of the constant region as a target antigen (PLPVIAELPPKVSVFVPPRDGFFGNP; SEQ ID NO:17). In addition, IgM-specific antibodies can be raised against isolated, intact Fc regions of the IgM protein, such Fc produced by trypsin cleavage at high temperature (Plaut and Tomasi, Proc. Natl. Acad. Sci. USA, 65:318-322, 1970).

### IgM-Binding Compounds

With the use of phage display technology, a peptide, YDWIPSSAW (SEQ ID NO:9), has been identified that binds with high affinity to murine IgM. This peptide also inhibits human rheumatoid factor (RF, mainly IgM class of anti-IgG autoimmune antibody) induced agglutination of IgG-coated latex beads, and shows no binding abilities to other immunoglobulins (Pati M. Glee et al. J Immunol, 1999, 163: 826-833).

Identifying Novel IgA- or IgM-Binding Compounds

The methods and compositions of the invention may also employ novel IgA-binding or IgM-binding compounds identified using techniques such as phage display. Phage display is a combinatorial screening technique, allowing the discovery and characterization of proteins that interact with a desired target by using multiple genes from a gene bank (George P. Smith, Science 228:1335, 1985). These genes represent a required diversity generated by DNA recombinant technology; therefore, each phage displays a unique random peptide. These genes are inserted into phages by replacing preexisting genes, thus creating a phage library. Premade libraries are readily available (e.g., Novagen T7Select) with accompanying kits. Novagen's T7 system is a lytic phage display that is preferred for cDNA libraries (J. Imm. Meth. 231:39; Nature Biotech. 19:1193), but other phages may also be used, such as non-lytic M13 bacterial filamentous phage, which is based on N-terminal fusion to surface coat proteins pIII and pVIII. The modified phages will then express the protein coded by the inserted DNA on its surface coat. In the case of M13, pIII display has a lower valency (1-5 per virion) and thus can be used to find high affinity compounds whereas pVIII has high valency (˜200 per virion) and can be used to find very low affinity binding compounds. Phage display services are commercially available through companies including Dyax Corp. of Cambridge, Mass., which offers four phage libraries: human antibody, proteins, structured peptides, and linear peptides. The phages from an entire phage library are incubated with the targeted proteins, (e.g., the alpha chain of IgA or the mu chain of IgM). Phages that display peptides with high affinity and specificity for IgA or IgM are then selected. The polynucleotide sequences coding for these peptides are identified and sequenced, and additional peptides can be produced for further analysis. The highest affinity and specificity protein, along with other qualities such as immunogencity, are then chosen as the final candidate to be used in the methods or compositions of the invention. Phage display gene banks or library may include millions of related genes, and thus may be employed to identify IgA- or IgM-binding compounds useful for the diagnostic methods and compositions of the invention.

### Polypeptide Expression

In general, polypeptides for use in the invention may be produced by any standard technique; for example, by transformation of a suitable host cell with all or part of a polypeptide-encoding polynucleotide molecule or fragment thereof in a suitable expression vehicle.

Those skilled in the field of molecular biology will understand that any of a wide variety of expression systems may be used to provide the recombinant polypeptide. The precise host cell used is not critical to the invention. A polypeptide for use in the invention may be produced in a prokaryotic host (e.g., E. coli) or in a eukaryotic host (e.g., Saccharomyces cerevisiae, insect cells, e.g., Sf21 cells, or mammalian cells, e.g., NIH 3T3, HeLa, or preferably COS cells). Such cells are available from a wide range of sources (e.g., the American Type Culture Collection, Rockland, Md.; also, see, e.g., Current Protocols in Molecular Biology, Eds. Ausubel et al., John Wiley and Sons). The method of transformation or transfection and the choice of expression vehicle will depend on the host system selected. Transformation and transfection methods are described, e.g., in Ausubel et al. (supra); expression vehicles may be chosen from those provided, e.g., in Cloning Vectors: A Laboratory Manual (Pouwels, P. H. et al., 1985, Supp. 1987).

One particular bacterial expression system for polypeptide production is the E. coli pET expression system (Novagen, Inc., Madison, Wis.). According to this expression system, DNA encoding a polypeptide is inserted into a pET vector in an orientation designed to allow expression. As the gene encoding such a polypeptide is under the control of the T7 regulatory signals, expression of the polypeptide is achieved by inducing the expression of T7 RNA polymerase in the host cell. This is typically achieved using host strains which express T7 RNA polymerase in response to IPTG induction. Once produced, recombinant polypeptide is then isolated according to standard methods known in the art, for example, those described herein.

Another bacterial expression system for polypeptide production is the pGEX expression system (Pharmacia). This system employs a GST gene fusion system which is designed for high-level expression of genes or gene fragments as fusion proteins with rapid purification and recovery of functional gene products. The polypeptide of interest is fused to the carboxy-terminus of the glutathione S-transferase protein from Schistosoma japonicum and is readily purified from bacterial lysates by affinity chromatography using Glutathione Sepharose 4B. Fusion proteins can be recovered under mild conditions by elution with glutathione. Cleavage of the glutathione S-transferase domain from the fusion protein is facilitated by the presence of recognition sites for site-specific proteases upstream of this domain. For example, polypeptides expressed in pGEX-2T plasmids may be cleaved with thrombin; those expressed in pGEX-3X may be cleaved with factor Xa.

Once the recombinant polypeptide is expressed, it is isolated, e.g., using affinity chromatography. In one example, an antibody (e.g., produced as described herein) raised against a polypeptide for use in the invention may be attached to a column and used to isolate the recombinant polypeptide. Lysis and fractionation of polypeptide-harboring cells prior to affinity chromatography may be performed by standard methods (see, e.g., Ausubel et al., supra).

Once isolated, the recombinant polypeptide can, if desired, be further purified, e.g., by high performance liquid chromatography (see, e.g., Fisher, Laboratory Techniques In Biochemistry And Molecular Biology, eds., Work and Burdon, Elsevier, 1980).

Polypeptides for use in the invention, particularly short peptide fragments, can also be produced by chemical synthesis (e.g., by the methods described in Solid Phase Peptide Synthesis, 2nd ed., 1984 The Pierce Chemical Co., Rockford, Ill.).

These general techniques of polypeptide expression and purification can also be used to produce and isolate useful peptide fragments or analogs (described herein).

The short peptides such as 50-mer Streptococcal IgA-binding peptide (Sap) and 58-mer modified Z-domain can also be chemically synthesized.

### Antibody Production

To generate antibodies, any standard technique may also be used. For example, a coding sequence for IgA1, IgM, or fragments thereof (e.g., the hinge region of IgA1, amino acids 217-241) may be chemically synthesized or expressed as a C-terminal fusion with glutathione S-transferase (GST) (Smith and Johnson, Gene 67:31-40, 1988). The fusion protein is purified on glutathione-Sepharose beads, eluted with glutathione, cleaved with thrombin (at the engineered cleavage site), and purified to the degree necessary for immunization of mice or rabbits. Primary immunizations are carried out with Freund's complete adjuvant or a similar adjuvant and subsequent immunizations with Freund's incomplete adjuvant. Antibody titres are monitored by ELISA or Western blot and immunoprecipitation analyses using the thrombin-cleaved polypeptide fragment of the GST fusion protein. Immune sera are affinity purified using CNBr-Sepharose-coupled polypeptide. Antiserum specificity is determined using a panel of unrelated GST proteins.

As an alternate or adjunct immunogen to GST fusion proteins, peptides corresponding to relatively unique immunogenic regions of IgA or IgM may be generated and coupled to keyhole limpet hemocyanin (KLH) through an introduced C-terminal lysine. Antiserum to each of these peptides is similarly affinity purified on peptides conjugated to BSA, and specificity tested in ELISA and Western blots using peptide conjugates, and by Western blot and immunoprecipitation using the polypeptide expressed as a GST fusion protein.

Alternatively, monoclonal antibodies which specifically bind IgA or IgM are prepared according to standard hybridoma technology (see, e.g., Kohler et al., Nature 256:495, 1975; Kohler et al., Eur. J. Immunol. 6:511, 1976; Kohler et al., Eur. J. Immunol. 6:292, 1976; Hammerling et al., In Monoclonal Antibodies and T Cell Hybridomas, Elsevier, N.Y., 1981; Ausubel et al., supra). Once produced, monoclonal antibodies are also tested for specificity by Western blot or immunoprecipitation analysis (by the methods described in Ausubel et al., supra). Antibodies which specifically recognize IgA, especially the IgA1 isotype, or IgM are considered to be useful in the invention. Alternatively monoclonal antibodies may be prepared using IgA or IgM and a phage display library (Vaughan et al., Nat. Biotechnol. 14:309-314, 1996).

Preferably, antibodies are produced using fragments of IgA or IgM which lie outside generally conserved regions (e.g., the 13 amino acid region in IgA1, absent in IgA2; see FIG. 1) or unique sequence in IgM hinge region; see SEQ ID NO:14 and appear likely to be antigenic by criteria such as high frequency of charged residues. In one specific example, such fragments are generated by standard techniques of PCR and cloned into the pGEX expression vector (Ausubel et al., supra). Fusion proteins are expressed in E. coli and purified using a glutathione agarose affinity matrix as described in Ausubel et al. (supra). To attempt to minimize the potential problems of low affinity or specificity of antisera, two or three such fusions are generated for each polypeptide, and each fusion is injected into at least two rabbits. Antisera are raised by serial injections, preferably including at least three booster injections.

Antibodies generated against IgA1 may be employed to diagnose IgAN and HSP, and antibodies anti-IgM may be used to diagnose IgMN.

### Peptide Modifications

It may be desirable to modify peptides used in the methods and compositions of the invention, as peptides that enter the circulation are rapidly degraded in plasma. Modifications are therefore desirable to prolong their half-life without reducing biological activity and binding specificity. For example, the tetradecapeptide somatostatin has only a 3 minute half-life in human plasma, far too short to be used as a radiopharmaceutical in cancer diagnosis. Thus, its analog, octreotide was developed to have a 90 minute half-life in the human circulation (Langer and Beck-Sickinger, Curr. Med. Chem. Anti-Cane. Agents 1:71-93, 2001). To resist degradation by exopeptidases, the peptides may be capped at the N- and/or C-terminus. This includes, for example, acetylation of the N-terminus, amidating or reducing the C-terminus, and N to C cyclization. These methods are standard in the art. To reduce susceptibility to endopeptidases the peptide may be modified by introducing D-amino acids, amino acid surrogates, or peptidomimetic sequences and spacers. Other ways to improve stability include modification of peptide bonds, replacement of amino- with amino-groups, methylation of amide nitrogens, and shortening of the natural amino acid sequence. Again, these methods are standard in the art.

### Radiometals

As indicated above, the diagnostic reagent further includes a detectable label, such as a radioactive label like a radioactive metal. Various radioactive metals are used in scintigraphy and their use varies with scintigraphy type, diagnosis being attempted, and cost and availability of the isotope. A key determinant in the isotope chosen is the scinitigraphy type, and radioactive decay needed. SPECT and PET use different isotopes because SPECT uses gamma emitters, whereas PET uses positron emitters. Isotopes can be produced in four different ways, which affects their cost. Those produced in a generator come from the conversion of a long half-life radioactive isotope to one of shorter half-life. This makes them practical for use even in small hospitals, and their cost is lower. Other radioactive metals are more expensive as they are produced in nuclear reactors, cyclotrons, and linear accelerators, and this may limit availability for smaller hospitals, and those in more remote areas. As for diagnosis, the radiometal used is in part dictated by the time needed to reach and to bind to the target. In the case of IgA-binding peptides, a longer half-life in the range of 4-80 hours may be preferred.

Metastable technetium-99m (^{99m}Tc) is used in 85% of scans, which is about 7 million each year in the U.S. alone. The properties of ^{99m}Tc are virtually ideal for diagnostic imaging; the γ-radiation of 140 keV falls within the ideal range of today's gamma cameras, and a half-life of 6 hours is long enough to synthesize the labeled radiopharmaceuticals, perform quality control, inject it into the patient, and carry out imaging. But the half-life is short enough to enable administration of sufficiently high doses with minimal patient risk, and at the concentration levels used (<10⁻⁶ M), neither the resulting gamma radiation nor the soft beta decay of ⁹⁹Tc is hazardous. ^{99m}Tc is readily available at low cost from its parent nuclide ⁹⁹Mo (t_{1/2} 66 h), produced in a ⁹⁹Mo/^{99m}Tc generator.

Gamma-emitting Indium-111 (¹¹¹In) has been extensively used for labeling monoclonal antibodies and peptides. The first peptide radiopharmaceutical approved for clinical use by the FDA was the ¹¹¹In-labeled somatostatin analog OctreoScan® (Mallinckrodt, St. Louis, Mo.). The chemistry and half-life of this isotope (67.9 h) makes it ideal for labeling immunoglobulins, where imaging may be performed several days after the initial injection. ¹¹¹In is produced in a cyclotron from Cadmium-111, and thus it is less available in comparison to ^{99m}Tc. Ion exchange and solvent extraction are the methods commonly used to separate Indium-111 from parent cadmium. However, for the purpose of radiolabeling an IgA-binding peptide, the longer half-life of ¹¹¹In compared to ^{99m}Tc provides the IgA-binding peptide more time to reach IgA deposits in the kidney and provides more time for clearance of circulating tagged IgA.

Galium-67 (⁶⁷Ga) is produced in a cyclotron from zinc-68 (⁶⁸Zn), and was the first nuclide produced for human use in 1953. The separation techniques may include solvent extraction and ion exchange, or both. The half-life of ⁶⁷Ga is over 78 hours, and its energy decay characteristics make it suitable for either gamma scintigraphy or PET imaging.

The radionuclides of copper offer a selection of diagnostic epitopes including ⁶⁰Cu, ⁶¹Cu, ⁶²Cu, and ⁶⁴Cu. The positron-emitting ⁶⁴Cu is cyclotron produced. ⁶⁴Cu is preferred for labeling proteins, peptides, and agents with long blood clearance, which is likely an advantageous property of IgA-binding proteins. ⁶⁴Cu has a half-life of 12.7 hours.

Tables 1 and 2 below show the many gamma- and positron-emitting radiometals for diagnostic use, their decay characteristics, half-life, and methods of production (Anderson and Welch, Chem. Rev. 99:2219-2234, 1999). Any of these radiometals may be used in the methods and compositions described herein.

TABLE 1
Gamma- and Beta-Emitting Radionuclides

| isotope | t_{1/2} (h) | production methods | decay mode | E_{γ} (keV) | E_{β}_{^{−}} (keV) |
|---|---|---|---|---|---|
| ⁶⁷Cu | 62.01 | accelerator, ⁶⁷Zn(n,p) | β⁻ (100%) | 91, 93, 185 | 577, 484, 395 |
| ⁶⁷Ga | 78.26 | cyclotron | EC (100%) | 91, 93, 185, 296 388 | |
| ⁹⁰Y | 64.06 | ⁹⁰Sr/⁹⁰Y generator | β⁻ (72%) | | 2288 |
| ¹¹¹In | 67.9 | cyclotron, ¹¹¹Cd(p,n)¹¹¹In | EC (100%) | 245, 172 | |
| ^{99m}Tc | 6.0 | ⁹⁹Mo/^{99m}Tc generator | IT (100%) | 141 | |
| ²⁰¹Tl | 72 h | cyclotron | EC (100%) | 135, 167 | |
| | | ²⁰³Tl(p,3n)²⁰¹Pb(p,n)²⁰¹Tl | Hg X-rays | | |

TABLE 2
Positron-Emitting Radionuclides

| | | | decay | |
|---|---|---|---|---|
| isotope | t_{1/2}(h) | methods of production | mode | E_{β}_{⁺} (keV) |
| ⁶⁵Co | 17.5 | cyclotron, ⁵⁴Fe(d,n)⁵⁵Co | β⁺ (77%) | 1513, 1037 |
| | | | EC (23%) | |
| ⁶⁰Cu | 0.4 | cyclotron, ⁶⁰Ni(p,n)⁶⁰Cu | β⁺ (93%) | 3920, 3000 |
| | | | EC (7%) | 2000 |
| ⁶¹Cu | 3.3 | cyclotron, ⁶¹Ni(p,n)⁶¹Cu | β⁺ (62%) | 1220, 1150 |
| | | | EC (38%) | 940, 560 |
| ⁶²Cu | 0.16 | ⁶²Zn/⁶²Cu generator | β⁺ (98%) | 2910 |
| | | | EC (2%) | |
| ⁶⁴Cu | 12.7 | cyclotron, ⁶⁴Ni(p,n)⁶⁴Cu | β⁺ (19%) | 656 |
| | | | EC (41%) | |
| | | | β⁻ (40%) | |
| ⁸⁹Ga | 9.5 | cyclotron, ⁶³Cu(α.nγ)⁶⁶Ga | β⁺ (56%) | 4150, 935 |
| | | | EC (44%) | |
| ⁶⁸Ga | 1.1 | ⁶⁸Ge/⁶⁸Ga generator | β⁺ (90%) | |
| | | | EC (10%) | 1880, 770 |
| ⁸²Rb | 0.022 | ⁸²Sr/⁸²Rb generator | β⁺ (96%) | 3150 |
| | | | EC (4%) | |
| ⁸⁶Y | 14.7 | cyclotron, ⁸⁶Sr(p,n)⁸⁶Y | β⁺ (33%) | 2335, 2019 |
| | | | EC (66%) | 1603, 1248 |
| | | | | 1043 |

### BFCAs

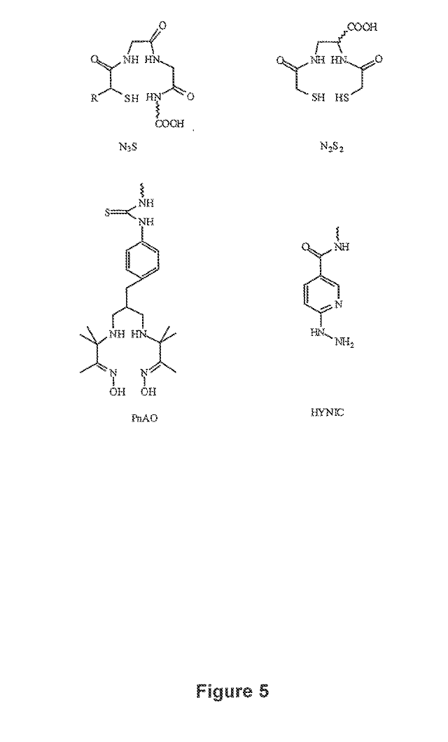

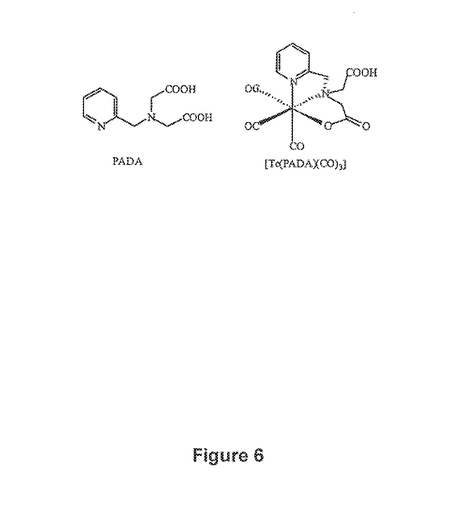

In radiopharmaceuticals, bifunctional chelating agents may be used to bridge the biological molecules and radiometals. There are many BFCAs available, any of which may be used in the invention. In one particular example, BFCAs that bind ^{99m}Tc have been most widely used because ^{99m}Tc is the metal used in 85% of the diagnostic scans currently in clinical use (Langer and Beck-Sickinger, Curr. Med. Chem. Anti-Canc. Agents 1:71-93, 2001). These BFCAs form a complex that has thermodynamic stability, kinetic inertness with respect to dissociation, and ability to stabilize the oxidation state of Tc. A variety of BFCAs have been developed for ^{99m}Tc including triamdiethiols N₃S, diamide-dithiols N₂S₂, propyleneamineoxime (PnAO), and hydrazinonicotinic acid (HYNIC). Some structures are shown in (FIG. 5). A newer approach employs an organometallic species [M(CO)₃(H₂O)₃]⁺ (M=⁹⁹Tc, ^{99m}Tc, ^{185/187}Re, ^{186/188}Re), which is a Tc/Re(I) precursor for labelling pharmacophores. The cationic species, available after short reaction times in high radiochemical purity, offer several advantages including maintenance of the oxidation state, stability in serum, and formation of stable complexes with biological ligands. Histidine and PADA (picolylamine-N,N-diacetic acid; FIG. 6) have been identified as suitable BFCAs. Histidine has been used to radiolabel a neurotensin derivative, and PADA for the labelling of bombesin- and neuropeptide Y derivatives with ^{99m}Tc.

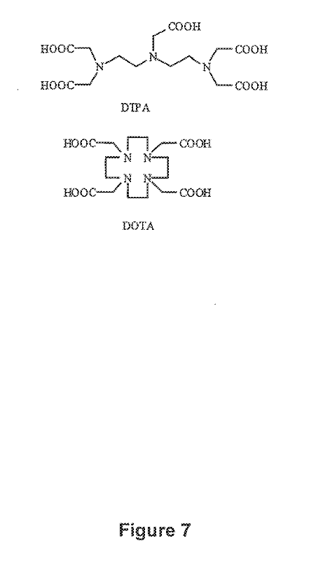

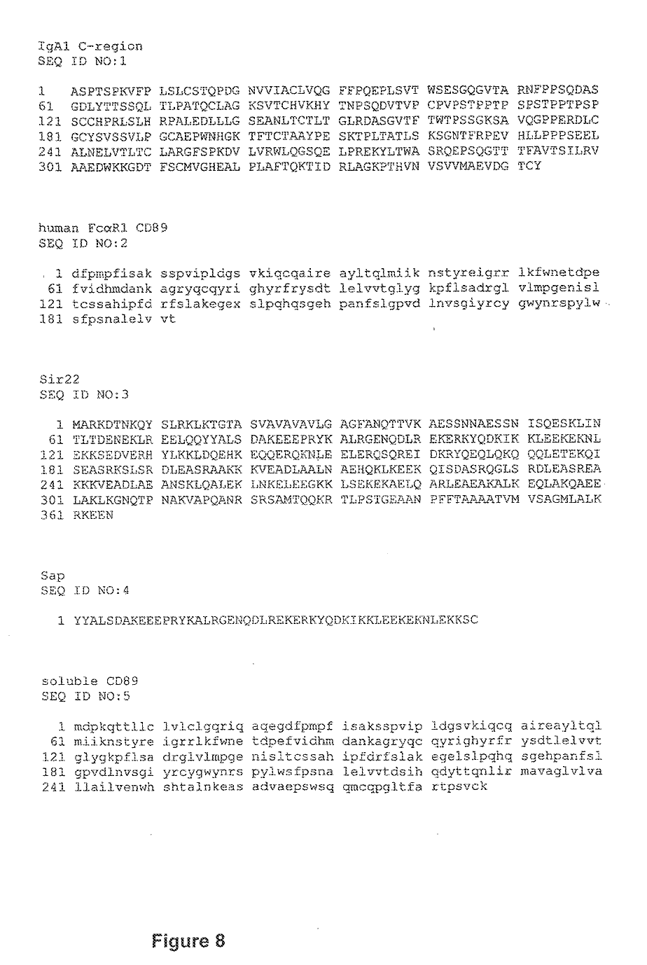

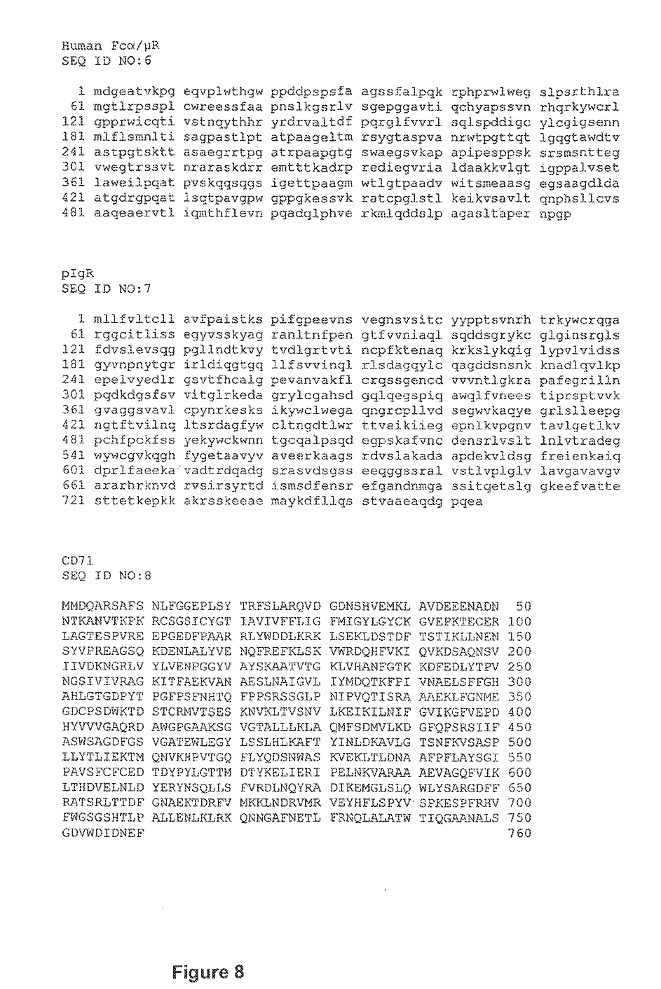

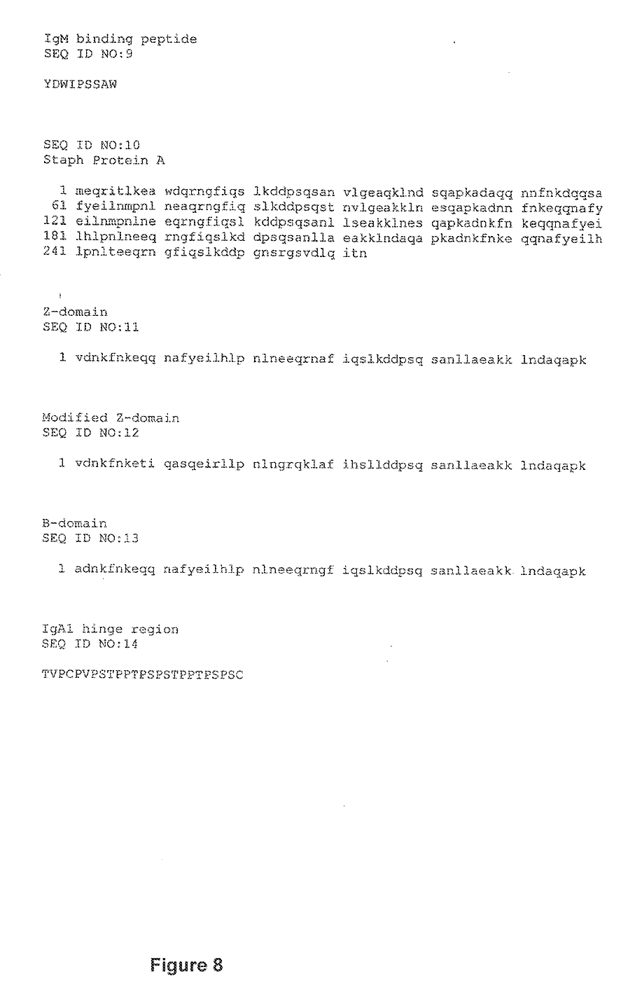

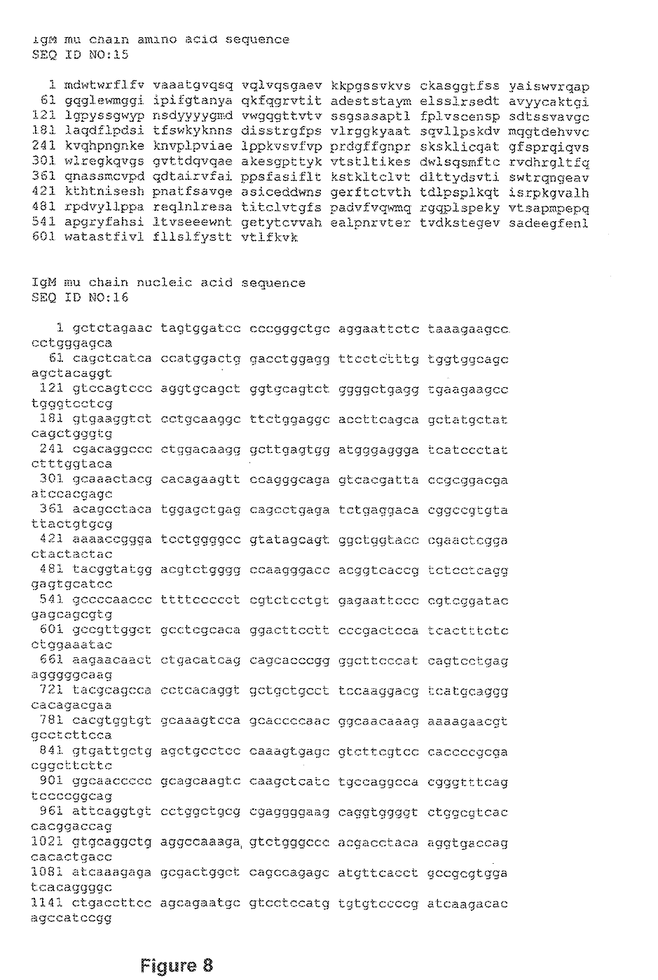

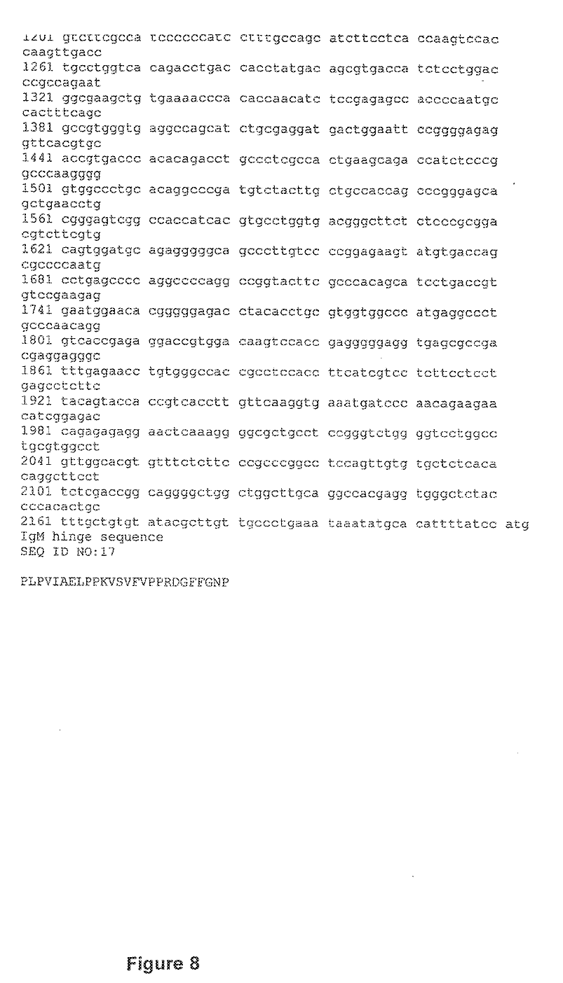

The BFCA of choice for Indium-111 is diethylene-triaminepentaacidic acid (DTPA), and a variety of peptides have been labeled with DTPA (FIG. 7). The second most widely used chelator for ¹¹¹In is DOTA (tetraazacyclododecanetetraacidic acid) (FIG. 7), which also can be used for complexing the therapeutic radionuclide Yttrium-90 (⁹⁰Y).

Tripeptides and tetrapeptides such as Lys-Gly-Cys, Cys-Gly-Cys, Gly-Gly-Cys, and Gly-Ala-Gly-Gly can also be used as BFCAs (Langer and Beck-Sickinger, Curr. Med. Chem. Anti-Canc. Agents 1:71-93, 2001). For example, ^{99m}Tc has been used to label vasoactive intestinal peptide (VIP), and its analog TP3654, which carries the chelating amino acid sequence GAGG on the C-terminus of VIP (Thakur et al., J. Nucl. Med., 41:107, 2000). In another report, ^{99m}Tc-labelling via N-terminal Ac-Cys-Gly-Cys-Gly (CGCG) chelation to α-melanocyte-stimulating hormone (MSH) resulted in promising candidates for peptide-based radio-diagnosis (Chen et al., Nucl. Med. Biol. 26:687-693, 1999).

### Non Radioactive Labels

In addition to radiolabeled compounds, paramagnetic substances and quantum dot (qdot) technology can also be used in the methods and compositions of the invention. Coupling paramagnetic substances to an IgA-binding or IgM-binding compound allows imaging via MRI, and qdot allows fluorescent imaging.

### Paramagnetic Substances

If an imaging technique (e.g., MRI) that does not require radioactivity is used in the present invention, a nonradioactive label may be substituted for the radiometal described above. For example, a paramagnetic substance (e.g., gadolinium) can be conjugated to an IgA-binding protein using methods standard in the art. When the compound is injected into a mammal and imaged by MRI, there is a difference in magnetic properties where the paramagnetic substances settle down. These differences can be used to identify IgA or IgM deposition in the glomeruli of the kidneys, and thus to diagnose an IgA or IgM kidney disease.

### Quantum Dots

Quantum dots (qdots) are fluorescent semiconductor nanocrystals made from variety of materials, for example CdS, CdSe, CdTe, CdHgTe/ZnS, InP, InAs, and PbSe. For nanocrystals smaller than the so-called Bohr exciton radius (a few nanometers), energy levels are quantized, with values directly related to the crystal size (an effect called quantum confinement). Thus, they have some distinguishing characteristics from commonly used fluorophores. One advantage of the qdots is that their size and shape can be precisely controlled by the duration, temperature, and ligand molecules used in the synthesis. The qdots' absorption and emission properties can also be controlled, because they are composition- and size-dependent.

The core of qdots may be wrapped by polymer coating materials to make them soluble in aqueous solutions and to prevent the release of cytotoxic Cd²⁺ or Se²⁻ ions from the qdot core. To add function to qdots, the outermost layer can include conjugated compounds (e.g., antibodies and bioactive peptides) that impart specific functionalities (e.g., IgA or IgM binding). If reduction of accumulation in liver and bone marrow is desired, high molecular weight polyethylene glycol (PEG) molecules can be added to the coating.

In one embodiment, qdots may be used alone for imaging purposes. Qdots tagged with antibodies (e.g., anti-IgA or anti-IgM antibodies) or binding compounds (e.g., IgA- or IgM-binding compounds) may be used to target the qdots to specific molecules. Such qdots can be injected into a mammal, and imaged using fluorescent techniques as described by Gao et al. (Nat. Biotech. 22:969-976, 2004).

In another embodiment, qdots may be coupled to radiolabeled compounds (e.g., the radiometals described herein) or paramagnetic substances (e.g., gadolinium). These qdots can be injected into a mammal, and visualized using an appropriate imaging technique (e.g., MRI, SPECT, PET, or planar scan).

### Formulation of Diagnostic Compositions

The compound(s) used in the compositions and methods the invention may be contained in any appropriate amount in any suitable carrier substance, and is generally present in an amount of 1-95% by weight of the total weight of the composition. The composition is preferably provided in a dosage form that is suitable for parenteral (e.g., intravenous) administration route. The diagnostic compositions may be formulated according to conventional pharmaceutical practice (see, e.g., Remington, The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York).

### Parenteral Compositions

The diagnostic compositions may be administered parenterally by injection, infusion, or implantation (intravenous or the like) in dosage forms, formulations, or via suitable delivery devices or implants containing conventional, non-toxic pharmaceutically acceptable carriers and adjuvants. The formulation and preparation of such compositions are well known to those skilled in the art of pharmaceutical formulation.

The composition may be in form of a solution, a suspension, an emulsion, an infusion device, or a delivery device for implantation, or it may be presented as a dry powder to be reconstituted with water or another suitable vehicle before use. Apart from the diagnostic compound(s), the composition may include suitable parenterally acceptable carriers and/or excipients. Furthermore, the composition may include suspending, solubilizing, stabilizing, pH-adjusting agents, tonicity adjusting agents, and/or dispersing agents.

As indicated above, the diagnostic compositions according to the invention may be in a form suitable for sterile injection. To prepare such a composition, the diagnostic compound(s) are dissolved or suspended in a parenterally acceptable liquid vehicle. Among acceptable vehicles and solvents that may be employed are water, water adjusted to a suitable pH by addition of an appropriate amount of hydrochloric acid, sodium hydroxide or a suitable buffer, 1,3-butanediol, Ringer's solution, dextrose solution, and isotonic sodium chloride solution. The aqueous formulation may also contain one or more preservatives (e.g., methyl, ethyl or n-propyl p-hydroxybenzoate). In cases where one of the compounds is only sparingly or slightly soluble in water, a dissolution enhancing or solubilizing agent can be added, or the solvent may include 10-60% w/w of propylene glycol or the like.

### Dosages of Compounds

While the attending physician ultimately will decide the appropriate amount of the compound or compounds for diagnosis of an IgA or IgM kidney disease, typically 1, 2, 3, 4, 5, 6, 7, 8, 9, or 10 mCi of a radiolabeled compound will be administered intravenously in a pharmaceutically acceptable carrier. Dosages are determined based on several considerations, including the dosage required for imaging technique (e.g., SPECT) used and by the clearance and absorption characteristics of the compound. Radioactive dosimetry can be used to ensure the maximum radiation allowed to each organ is not exceeded, based on published values.

### Imaging Techniques

Any standard imaging technique can be used in the present invention, with scintigraphy being the preferred method. Those skilled in the art will know which types of scanner are used with specific radiographic agents.

### Scintigraphy

Scintigraphy involves the use of radioactive isotopes to diagnose and treat various diseases. It has applications in neurology, cardiology, oncology, endocrinology, lymphatics, urinary function, gastroenterology, pulmonology, and other areas. Generally, the radioactive isotope is chemically bonded to a specific compound, and then injected intravenously. Different radioactive isotopes are used for different scintigraphy methods and three common methods are SPECT, PET, and planar scan (Anderson and Welch, Chem. Rev. 99:2219-2234, 1999; Langer and Beck-Sickinger, Curr. Med. Chem. Anti-Canc. Agents 1:71-93, 2001).

SPECT (Single Photon Emission Computer Tomography) is a nuclear imaging technique that uses gamma rays. After injection of an isotope, a rotating gamma camera collects images 360 degrees around a patient, selecting only photons of certain energy. The images from various levels of the body are processed and recombined to form a 3D image.

PET (Positron Emission Tomography) is a nuclear imaging technique that uses radioactive isotopes that decay by positron emission, resulting in the release of two photons in opposite directions. A 360-degree scanner detects these photons, but only paired photons are processed. The safety of PET scans is higher than body CT, with radiation absorbed from a PET scan averaging 5 mSv, whereas the radiation absorbed from a CT scan of the body ranges from 6-16 mSv.

Planar scanners were among the first generation of scintillation detecting devices and are currently used in many diagnostic procedures. They are in common use and yield two-dimensional images.

### MRI

In addition to scintigraphy methods, magnetic resonance imaging (MRI) can also be used in the methods of the invention. MRI uses strong magnetic fields to generate three dimensional tomographic images of patients' bodies and does not require radioactivity.

### Radiological Optimization

To further optimize the diagnostic methods of the invention, renal tubule retention may be reduced and pretargeting strategies employed.

### Renal Tubule Retention of Radioactive Chelates

Radiolabeled peptides, proteins, or their fragments smaller than approximately 60 kDa are filtered at the glomerulus and enter the renal tubule (Langer and Beck-Sickinger, Curr. Med. Chem. Anti-Canc. Agents 1:71-93, 2001; Thakur et al., J. Nucl. Med., 41:107, 2000). Under physiological conditions, the cells of the proximal tubule quantitatively reabsorb these peptides, which are then subject to lysosomal degradation. This reabsorption traps the radiometal chelate in the cell, resulting in high retention of the radiolabel in the renal medulla and reducing the scintigraphic sensitivity for detection of specific higher emissions from the medullary region of the kidney. However, in IgAN, IgA deposits are in the glomeruli located in the renal cortex, minimizing the problem of renal radiometal retention and the associated interference. Reduction of renal tubule retention in diagnosing an IgA or IgM kidney disease may still be desirable, and can be accomplished by using more lipophilic molecules. These molecules have a high content of lipophilic amino acids in the peptide, or have the addition of a fatty acyl moiety on the molecule. In one example, such lipophilic modification can be seen in RC-160, a somatostatin analog with increased lipophilicity that has fewer tendencies towards renal excretion (P. Dasgupta and R. Mukherjee, Br J Pharmacol (2000) 129, 101-109). A second example is stearyl-Nle¹⁷-VIP, a lipophilic analogue of 28-mer vasoactive intestinal peptide, VIP. (Gozes et al., J. Pharmacol. Exper. Therap. 273 (1995) 161-167). The lipophilic modification may promote metabolic degradation through the liver, rather than the kidney. Another way to reduce renal tubular accumulation of radiometals is to increase the size of the radiolabeled reagent to be large enough to avoid filtration through glomeruli (Langer and Beck-Sickinger, Curr. Med. Chem. Anti-Canc. Agents 1:71-93, 2001). A third method is to administer oral or injected basic amino acids (e.g., lysine) or their derivatives following administration of the diagnostic agent, thereby inhibiting renal tubular cell uptake of proteins and peptides and inducing a transient proteinuria (Behr et al., Eur. J. Nucl. Med. 25:201-212, 1998).

### Pretargeting Strategies

Pretargeting delivery is commonly used in tumor radioimmunoscintigraphy (RIS) and radioimmunotherapy (RIT) (Anderson and Welch, Chem. Rev. 99:2219-2234, 1999; Langer and Beck-Sickinger, Curr. Med. Chem. Anti-Canc. Agents 1:71-93, 2001; Chang et al., Mol. Canc. Ther. 1:553-563, 2002; Rossi et al., Clin. Canc. Res. 9:3886s-3896s, 2003). Pretargeting reduces or clears unbound radiolabeled molecules in the circulation, at the time when bound concentration is highest. The most widely used system is the biotin-streptavidin (SA) pair. Generally, the non-radiolabeled mAb-SA is injected first. As the clearance of antibodies is slow in circulation, unbound antibodies need to be cleared before delivering radiolabeled biotin. Thus, after 1-2 days of the first injection, a galactosylated clearing agent that interacts with mAb-SA is injected. This agent quickly clears away any circulating mAb-SA, but it is unable to clear bound mAb-SA. After another 1-10 hours, the radiolabeled biotin-BFCA is injected. Therefore, the radioactive chelate is only delivered to bound locations instead of to the circulating antibodies. Other possible strategies include a similar approach with a binding pair other than biotin-streptavidin or a system that requires only two steps, for example, as described below.

### Two Step Method

To reduce the intravascular IgA or IgM radioactivity background, a two step method may also be utilized. In this method, an anti-IgA1 antibody or an anti-IgM antibody is first prepared, from which an Fab domain is obtained by limited proteolysis. This Fab is then modified to include streptavidin (SA) and galactose (Gal). The streptavidin is added as a biotin ligand, and Gal is added to promote removal of circulating Fab-IgA1 or Fab-IgM complexes. As clearance of the SA-Gal-Fab by hepatic ASGPR (asialoglycoprotein receptors) is faster than binding to IgA or IgM, a suitable competitive inhibitor such as galactose-Ficoll, a synthesized polymer of galactose that effectively inhibits ASGPR function, is injected (Rifai et al., J. Exp. Med. 191:2171-2181, 2000). Simultaneously with or immediately following the galactose-Ficoll injection, the SA-Gal-Fab anti-IgA1 or SA-Gal-Fab anti-IgM, is injected into patients for pretargeting, binding to both circulating IgA1 and IgA1 deposited in the renal cortex, or circulating IgM and IgM deposited in the renal cortex. At 24-48 hours, when the effect of galactose-Ficoll ends, the circulating SA-Gal-Fab-IgA1 or SA-Gal-Fab-IgM complexes are cleared from the circulation via the hepatic ASGPR. However, the immobilized SA-Gal-Fab-IgA1 or SA-Gal-Fab-IgM complexes in the glomeruli persist, and are available for imaging.

Approximately one or two days after the first injection, with background vascular SA-Gal-Fab-IgA1 or SA-Gal-Fab-IgM complex substantially cleared, radiolabeled (^{99m}Tc or ¹¹¹In) biotin-HSA-Gal (biotinylated human serum albumin conjugated with galactose) is injected and binds to SA-Gal-Fab-IgA1 SA-Gal-Fab-IgM complexes in the kidney. After another 4-24 hours, the removal of unbound (free) radiolabeled Biotin-HSA-Gal from the circulation by hepatic ASGPR results in increased target to noise ratio in the renal cortex. In addition, albumin's size (66 kDa) excludes the radioactive complex from being filtrated from glomeruli, thus reducing associated background radiation due to settling of radioactive chelates in the proximal tubule cells of the kidneys. Another advantage of this design is that streptavidin is a tetramer (MW: 4×13 kDa=52 kDa), binding 4 molecules of biotinylated ligands. This increases the imaging signal, with four moles of radioactive Biotin-HSA-Gal binding per mole streptavidin exposed in the renal cortex.

To carry out this method, anti-human IgA1 antibody or anti-human IgM antibody and its Fab fragment are produced to diagnose an IgA or IgM kidney disease. Mouse or chimeric monoclonal anti-human IgA1 hinge region or anti-human IgM antibodies are raised by conventional methods, for example, as described herein. The Fab fragment is then obtained by any standard technique, such as papain digestion, and then purified, for example, by gel-filtration chromatography followed by antigen-affinity chromatography.

To conjugate galactose molecules onto Fab, cyanomethyl-2,3,4,6-tetra-I-acetyl-1-thio-β-D-galactopyranoside (C-4141; Sigma) is dissolved in methanol at 0.1 M and mixed with 0.1 volume sodium methoxide (methanol sodium derivative; J.T. Baker Chemical Co., Phillipsburg, N.J.). After 48 h at room temperature, this mixture is stored for weeks at 4° C. The conjugate is replaced in 0.25 M sodium borate buffer, pH 8.5, containing Fab at 1.0 mg,/ml. After 2 h at room temperature, the sample is dialyzed into phosphate-buffered saline. The antigen-binding function of the antibody is not altered by the conjugation (Ong et al., Canc. Res. 51:1619-1626, 1991).

Streptavidin (SA; MW: ˜52 kDa) binds specifically with biotin (244 Da). It is derived from the bacterium Streptomyces avidinii and bears similarity to chicken egg-white avidin both in three-dimensional structure and its ability to bind biotin with extremely high affinity (Kd=10⁻¹⁵ M). It is a tetrameric protein (4×13 kDa) capable of binding up to 4 biotin molecules. Unlike avidin, streptavidin is non-glycosylated and is essentially neutral in charge, whereas avidin (pI˜10.5) is basic at neutral pH. Because of this, streptavidin has considerably less non-specific binding resulting in less background and has replaced avidin as the preferred reagent for applications where protein interactions may cause undesired background.

To conjugate SA to Gal-Fab, the Gal-Fab is chemically conjugated to SA (Genzyme, Cambridge, Mass.) using succinimidyl 4-(N-maleimido-methyl) cyclohexane-1-carboxylate (SMCC). Excess SMCC is offered to SA in a 3:1 molar ratio in sodium borate at pH 8 containing 5% DMSO. After 30 min, the SMCC-SA is desalted by Sephadex G-25 (Amersham Pharmacia) gel filtration. The Gal-Fab is reduced with DTT, desalted by gel filtration and mixed in 1:1 molar ratio with SMCC-SA at 5 mg/ml total protein concentration in PBS. The reaction is monitored by size exclusion HPLC, and after sufficient conjugate has formed, ca. 50 min, the reaction is quenched by the addition of sodium tetrathionate to 5 mM to reoxidize unreacted thiols. The conjugate is separated from free SA by Q Sepharose chromatography. The conjugate is also separated from unconjugated Gal-Fab by affinity chromatography using immobilized iminobiotin (Pierce) equilibrated in 0.05 M sodium carbonate, 0.5 M sodium chloride, pH 11 and eluting with 0.05 M sodium acetate/0.5 M sodium chloride, pH 4. About 80% of the conjugate consists of SA/antibody in a 1:1 ratio, with the remainder primarily in a 2:1 ratio or higher (Axworthy et al., Proc. Natl. Acad. Sci. U.S.A. 97:1802-1807, 2000).

HSA (human serum albumin) is a single polypeptide chain protein containing 584 amino acids (66 kDa), and is the most abundant protein in the blood, accounting for about 60% of protein in the plasma. HSA is exclusively produced in liver, and unlike most other plasma proteins, it contains no carbohydrates. HSA has a half-life of about 19 days. The main functions of HSA include maintenance of colloidal osmotic pressure in the blood vessels, contribution to maintenance of acid/base metabolism, and transport of intrinsic substances and medications. Highly purified, virus free, recombinant human albumin in pharmaceutical excipient quality can be obtained commercially (e.g., GTC Biotherapeutics).

To prepare biotin-HSA-Gal, HSA is combined with 3-fold excess N-hydroxysuccinimide-LC-biotin (Pierce) in 0.5 M sodium borate (pH 8.5), 5% DMSO. After ca. 4 hr the solution is added to a 200-fold excess of freshly prepared neat 2-imino-2-methoxyethyl 1-thio-β-D-galactopyranoside. The mixture is stirred for 8 hr at room temperature and purified by diafiltration into PBS. The final material contains 1.6 moles of biotin per mole of HSA as determined by displacement of 2-(4′-hydoxyphenylazo)-benzoic acid (HABA) from SA and 30-40 moles of thiogalactose per mole of HSA as determined by a colorimetric anthrone assay (Axworthy et al., Proc. Natl. Acad. Sci. U.S.A. 97:1802-1807, 2000).

To conjugate bifunctional chelating agent (BFCA) DOTA (1,4,7,10-tetraazacyclododecane-1,4,7,10-tetraacetic acid) onto biotin-HSA-galactose, the COOH group of DOTA is activated by a carbodiimide reagent. Briefly, DOTA is dissolved in anhydrous DMSO at 80° C., and the solution is cooled under argon. A solution of N-hydroxy-2,5-pyrrolidinedione in DMSO is added dropwise to a stirred solution of DOTA, followed by the dropwise addition of N,N′-dicyclohexylcarbodiimide in DMSO. The molar ratio between DOTA:N-hydroxy-2,5-pyrrolidinedione:N,N′-dicyclohexylcarbodiimide is 1:1.4:0.8. The reaction mixture is stirred overnight and then filtered to separate a by-product, dicyclohexylurea. The conjugation between DOTA and biotin-HSA-Gal is carried out at a molar ratio of 50:1 by adding an adequate volume of the DOTA-activated ester solution to the biotin-HSA-Gal dissolved in 0.1 M phosphate buffer (pH 8.0). After the overnight reaction, the conjugate is purified by a reverse-phase column in a FPLC system coupled with a UV detector and a radiodetector. A linear gradient method is applied using an aqueous 0.1% trifluoroacetic acid solution (solvent A) and methanol (solvent B). The eluents are delivered at a flow of 4 ml/min, starting from 0% of solvent A to 100% of solvent B in 37 ml. Two peaks corresponding to the Biotin-HSA-Gal conjugates with DOTA are observed in the UV profile. The retention volume for biotin-HSA-gal conjugates with DOTA differ from that of unconjugated biotin-HSA-gal. An integrated fraction collector is used to recover each compound and further analysis by MALDI-TOF (matrix-assisted laser desorption/ionization combined with time-of-flight) mass spectrometry (Bussolati et al., Canc. Res. 61:4393-4397, 2001) can be performed if desired.

To conjugate with the radiolabel, indium-111-chloride (¹¹¹InCl₃) with no carrier added is obtained from, for example, Mallinckrodt, Inc. (St. Louis, Mo.). DOTA-biotin-HSA-galactose conjugate is dissolved in 0.2 M ammonium acetate buffer (metal free; pH 7), and then incubated for 30 min at 100 C with ¹¹¹InCl₃ at a ratio of about 20 MBq ¹¹¹In/l nmol conjugate. The purity of radiolabeled conjugate is assessed by instant thin layer chromatography (ITLC). The ITLC system uses an ITLC-SG (silica gel) support (Gelman Sciences, Ann Arbor, Mich.) and 4 mM EDTA (pH 4.0) as a mobile phase. The peptide-bound activity remains at the origin, and the previously uncomplexed radiometal moves at the solvent front as an EDTA complex. The radiolabeling efficiency is typically greater than 97% and is critically dependent on the chemical purity (metal cations) of the ¹¹¹InCl₃ (Smith-Jones et al., Endocrinology 140:5136-5148, 1999).

Galactose-Ficoll can be readily synthesized as follows. Cyanomethyl 1-thio-β-D-galactopyranoside is dissolved in methanol at an approximate ratio of 1.0 mmol per 10 ml, with the addition of 0.5 ml of 0.5 M sodium methoxide in methanol. After 48 hours of stirring at room temperature in a tightly capped glass vial, the methanol will be evaporated with a stream of nitrogen. To the dried residue is added 1 gram of Ficoll 70 or Ficoll 400 from Pharmacia or other manufacturers in 100 ml BBS (0.16 M sodium chloride-0.2 M sodium borate, pH 8.0). This is stirred at room temperature for 24 hours, then the reaction is stopped by adding 10 ml of 1.0 N acetic acid. The reaction mixture will be dialyzed exhaustively against distilled water (Plotz and Rifai, Biochemistry 1982, 21, 301-308; Lee at el. Biochemistry 1976, 15, 3956-3963).

Administration of the compounds of the invention may be intravenous. In one example, 2.0 mCi of ¹¹¹In is injected into patients intravenously over a period of 30-60 seconds. Images are taken 24-48 hours post injection as described below; however, the time between administration and imaging may be altered as desired or based on clearance and retention characteristics of the particular compounds used in the diagnostic method.

Any of the several SPECT scanners commercially available may be used in this method (e.g., the GE MyoSIGHT, General Electric Company). One skilled in the art will know what settings (e.g., collimators, window size, and energies) can be used with specific radiometals or isotopes. In one example, a medium energy collimator is used for ¹¹¹In, with symmetric windows of 15-20% set at the 173 and 247 keV (the photopeaks of ¹¹¹In). In another example, a low energy collimator is used for ^{99m}Tc, with symmetric windows of 20% set at 140 keV. The number of images to be taken depends on the capabilities of the scanning system, and the desired image quality. A64×64 matrix set of images may be taken, usually with the camera moving 360 degrees with 64 stops. If higher image quality is desired, a 128×128 or 256×256 matrix set of images may be taken. The scan may take 30-60 minutes during which time the patient must remain absolutely still. Additional patient preparation (e.g., oral hydration, cathartics, and enemas), if desired, may be performed to obtain optimal images.

The images can be used to determine the amount of radioactivity in the kidney of the patient. By comparing this amount of radioactivity to the radioactivity in the kidney of a patient known not to have IgA nephropathy or HSP, an increase in radioactivity detected in the kidney is diagnostic of IgA nephropathy or HSP.

Similarly, in the case where anti-human IgM is used in place of anti-human IgA, an increase in the amount of radioactivity determined from the images relative to the radioactivity in the kidney of a patient known not to have IgM nephropathy is diagnostic of IgM nephropathy.

### OTHER EMBODIMENTS

All publications, patent applications including U.S. provisional application No. 60/705,282, filed Aug. 3, 2005, and patents mentioned in this specification are herein incorporated by reference.

Various modifications and variations of the described method and system of the invention will be apparent to those skilled in the art without departing from the scope and spirit of the invention. Although the invention has been described in connection with specific desired embodiments, it should be understood that the invention as claimed should not be unduly limited to such specific embodiments

What is claimed is:

1. A method for diagnosing an IgA or IgM kidney disease in a mammal, said method comprising:
(a) administering to said mammal a compound which specifically binds IgA or IgM; and
(b) detecting said compound in the kidney of said mammal, wherein an increase in the amount of said compound in said kidney of said mammal relative to the amount in the kidney of a mammal without said kidney disease is diagnostic of said IgA or IgM kidney disease in said mammal.

2-3. (canceled)

4. The method of claim 1, wherein said compound comprises a peptide.

5. The method of claim 4, wherein said peptide is selected from the group consisting of human FcαR1 (SEQ ID NO:2), human Fcα/μ receptor (SEQ ID NO:6), polymeric Ig receptor (SEQ ID NO:7), Sir22 (SEQ ID NO:3), streptococcal IgA-binding peptide (Sap; SEQ ID NO:4), a modified Z-domain protein (SEQ ID NO: 12), and YDWIPSSAW (SEQ ID NO:9), or an IgA- or IgM-binding fragment thereof.

6. (canceled)

7. The method of claim 1, wherein said compound comprises an antibody, or an IgA- or IgM-binding fragment thereof.

8-16. (canceled)

17. The method of claim 1, wherein said compound is linked to a paramagnetic substance.

18. The method of claim 17, wherein said paramagnetic substance is gadolinium.

19-20. (canceled)

21. The method of claim 1, wherein said compound further comprises a galactose and a first member of a binding pair.

22. The method of claim 21, wherein said first member of a binding pair is streptavidin.

23. The method of claim 21, wherein said administration further comprises administration of galactose-Ficoll.

24. The method of claim 21, wherein said detection is performed by administering to said mammal a radioactively labeled compound conjugated to both (a) a second member of a binding pair and (b) a galactose, followed by detecting said radioactively labeled compound in said kidney of said mammal.

25-27. (canceled)

28. A composition comprising:
(a) an IgA- or IgM-binding compound;
(b) a bifunctional chelating agent; and
(c) a detectable label in a pharmaceutically acceptable carrier, wherein said IgA- or IgM-binding compound is linked to said detectable label through said bifunctional chelating agent.

29. The composition of claim 28, wherein said compound comprises a peptide.

30. The composition of claim 29, wherein said peptide is selected from the group consisting of human FcαR1 (SEQ ID NO:2), human Fcα/μ receptor (SEQ ID NO:6), polymeric Ig receptor (SEQ ID NO:7), Sir22 (SEQ ID NO:3), streptococcal IgA-binding peptide (Sap; SEQ ID NO:4), a modified Z-domain protein (SEQ ID NO: 12), and YDWIPSSAW (SEQ ID NO:9), or an IgA- or IgM-binding fragment thereof.

31. (canceled)

32. The composition of claim 28, wherein said compound comprises an antibody, or an IgA- or IgM-binding fragment thereof.

33-37. (canceled)

38. The composition of claim 28, wherein said detectable label is a radioactive label.

39. (canceled)

40. A kit comprising:
(a) an IgA- or IgM-binding compound;
(b) a bifunctional chelating agent; and
(c) a detectable label in a pharmaceutically acceptable carrier,
(d) instructions for use for detecting an IgA or IgM kidney disease in a mammal.

41. The kit of claim 40, wherein said compound further comprises a galactose and a first member of a binding pair; and said detectable label comprises a radioactively labeled peptide, said radioactively label peptide comprising a galactose and a second member of a binding pair.

42. The kit of claim 41, wherein said radiolabel is selected from the group consisting of ^{99m}Tc, ¹¹¹In, ⁶⁶Ga, ⁶⁷Ga, ⁶⁸Ga, ⁸⁶Y, ⁹⁰Y, ^{201 Tl,} ⁵⁵Co, ⁶⁰Cu, ⁶¹Cu, ⁶²Cu, ⁶⁴Cu, ⁶⁷Cu, ⁸²Rb, ^{185/187}Re, and ^{186/188}Re.

43. The kit of claim 41, wherein said first member of a binding pair is streptavidin, and said second member of a binding pair is biotin.

44. The kit of claim 41, wherein said radioactively labeled peptide is human serum albumin.
